(19) United States
(12) Patent Application Publication
(10) Pub. No.: US 2025/0249216 A1
(43) Pub. Date: Aug. 7, 2025

(54) EXPANDABLE ACCESS SHEATH AND RELATED DEVICES

(71) Applicant: Cultiv8 Medical, LLC, Minneapolis, MN (US)

(72) Inventors: Bryan Cheng Yang GOH, Minneapolis, MN (US); Gregg Stuart SUTTON, Maple Grove, MN (US); Peter T. Keith, St. Paul, MN (US); Stephen P. Michael, Maple Grove, MN (US); Mongkot LOR, Brooklyn Park, MN (US)

(73) Assignee: Cultiv8 Medical, LLC, Minneapolis, MN (US)

(21) Appl. No.: 19/047,817

(22) Filed: Feb. 7, 2025

Related U.S. Application Data
(60) Provisional application No. 63/751,413, filed on Jan. 30, 2025.
(60) Provisional application No. 63/711,027, filed on Oct. 23, 2024.
(60) Provisional application No. 63/664,395, filed on Jun. 26, 2024.
(60) Provisional application No. 63/560,332, filed on Mar. 1, 2024.
(60) Provisional application No. 63/550,924, filed on Feb. 7, 2024.

(51) Int. Cl. A61M 25/06; A61M 25/00; A61M 25/01

(52) U.S. Cl. CPC A61M 25/0662; A61M 25/0023; A61M 25/005; A61M 25/0074; A61M 25/0102; A61M 2025/0024; A61M 2025/0681; A61M 2025/0687

(57) ABSTRACT

Expandable vascular introducer sheath devices, systems and method for low profile delivery, expansion to accommodate intravascular devices, and recovery for subsequent removal. The expandability features may be incorporated into other intravascular devices such as guide extension catheters, thrombus removal devices, etc.

### CROSS-REFERENCE TO RELATED APPLICATIONS

This application claims the benefit of U.S. Provisional Patent Application No. 63/751,413, filed Jan. 30, 2025; U.S. Provisional Patent Application No. 63/711,027, filed Oct. 23, 2024; U.S. Provisional Patent Application No. 63/664,395, filed Jun. 26, 2024; U.S. Provisional Patent Application No. 63/560,332, filed Mar. 1, 2024; and U.S. Provisional Patent Application No. 63/550,924, filed Feb. 7, 2024, the entire disclosures of which are incorporated herein by reference.

This application is related to U.S. Non-Provisional patent application Ser. No. 18/644,816, filed Apr. 24, 2024, the entire disclosure of which is incorporated herein by reference.

### TECHNICAL FIELD

The present disclosure generally relates to introducer sheath systems that are intended to be inserted into the vasculature to provide a conduit for introducing intravascular devices while providing a hemostatic seal to minimize blood loss. The embodiments of the present disclosure also have application to other catheter and cannula constructions and other associated clinical applications.

### BACKGROUND

Vascular access sheaths are used to establish an entry point and conduit into a blood vessel, such as a femoral, radial or jugular artery. Once in position, subsequent catheters or devices can be introduced in the vascular access sheath. Vascular access sheaths are typically uniform diameter and non-expandable. Conventional non-expandable introducer sheaths are typically formed from an elongate tubular shaft of uniform diameter, with a proximal hub, containing a hemostatic valve and flushing side port. During introduction into the blood vessel, an elongate dilator and guidewire are typically used.

Although non-expandable introducer sheaths have great utility, there are clinical situations where expandability is useful. Expandable introducer sheaths have been used in large bore applications such as delivering TAVR devices. Such sheaths are typically 14 F or greater and expand to 18 F or more. Examples of such large bore expandable sheaths include the Solopath™ sheath by Terumo, the iSleeve™ sheath by Boston Scientific, and the eSheath™ by Edwards Lifesciences. These expandable introducer sheaths have performance limitations, particularly related to a recovery profile after expansion. Further, they have design limitations particularly related to wall thickness and construction, limiting their application to large bore procedures, such as 12 F or more.

### SUMMARY OF THE DISCLOSURE

Thus, there is a need for an expandable sheath suitable for medium bore (e.g., <12 F) and small bore (e.g., <8 F) procedures. For example, for radial procedures, where the radial artery at the access site is about 1.8 mm to 2.3 mm (5.4 F to 6.9 F) in outside diameter, small bore sheaths are typically used, such as 4 F to 6 F, sometimes 7 F. The radial artery is particularly prone to spasm. Spasm can cause significant patient pain, and if severe enough, the radial artery can “clamp down” enough on the sheath (and other instruments) that they can be difficult to remove from the patient.

The mechanisms that provoke spasm are not well defined, but are thought to be triggered by mechanical irritation of the vessel, such as scraping (particularly the intima), repeated contact, overexpansion, etc. Similarly, the same irritation may cause the formation of a thrombus, potentially leading to radial artery occlusion (RAO). In an effort to mitigate and manage spasm and RAO, efforts such as coating sheaths with a lubricious hydrophilic coating, and injection of antispasmodic medications have helped, but the problem persists.

One approach to minimizing spasm and its effects is to utilize inventive expandable sheaths as described here. Such expandable sheaths are lower profile than conventional sheaths upon introduction and retraction. The lower profile (reduced diameter) is less forceful against the inside surface of the radial artery, both because the diameter is smaller, and also because the reduced diameter yields a more flexible device.

Once positioned, such sheaths would achieve their full diameter by diametric radial expansion brought about by passage of a dilator or other catheter devices inserted therein. Such catheters could be coronary guide catheters, guide wires, diagnostic catheters, or other catheter like devices. Radial diametric expansion is thought to be a less traumatic way to achieve a large luminal diameter therethrough, versus introducing a full-sized sheath and frictionally engaging or scraping along the interior of the radial artery. In some instances, such an expandable sheath would recover to a smaller diameter upon retraction of the prior placed devices within its lumen. In this manner, the retraction of the sheath from the patient would also be less traumatic, as its engagement with the inner surface of the radial artery is minimized.

The embodiments and concepts described herein may also have application to other devices, such as large bore introducer sheaths, guide catheter extensions, etc.

Such embodiments are described in more detail hereinafter with reference to the drawings. The above summary is not intended to describe each and every embodiment or implementation of the present disclosure, and is not intended to limit the disclosure.

### BRIEF DESCRIPTION OF THE DRAWINGS

The drawings illustrate example embodiments of the present disclosure and, along with the description, serve to explain the principles of the disclosure. The drawings are only illustrative of certain embodiments and do not limit the disclosure or invention.

FIG. 1 is a schematic side view of an introducer sheath according to an embodiment of the present disclosure.

FIG. 2 is an enlarged schematic view of a portion of the introducer sheath shown in FIG. 1.

FIG. 3 is a cross-sectional view a portion of the introducer sheath shown in FIG. 2.

FIG. 4 is a cross-sectional view a portion of the introducer sheath shown in FIG. 5.

FIG. 5 is s an enlarged schematic view of a portion of the introducer sheath shown in FIG. 1, showing the flaps tucked.

FIGS. 6 and 7 are schematic cross-sectional views of alternative edge configurations.

FIG. 8 is a schematic side view of the introducer sheath shown in FIG. 1, together with a setting tube.

FIG. 9 is a schematic side view of the introducer sheath shown in FIG. 1, illustrating a pucker or tent.

FIG. 10 is a schematic side view of the introducer sheath shown in FIG. 9, illustrating a uniform outside profile where the pucker or tent was reformed.

FIGS. 11-15B are schematic illustrations of various alternative embodiments of distal flap edges.

FIGS. 16-19 are schematic illustrations of various alternative embodiments incorporating an elastomeric jacket.

FIG. 20 is a schematic side view of an introducer sheath according to an alternative embodiment of the present disclosure.

FIGS. 21 and 22 are cross-sectional schematic views showing an embodiment with angled edges at the slit.

FIGS. 23-25 are schematic partial side views showing various dilator configurations.

FIGS. 26, 28 and 30, and their corresponding cross-sectional views shown in FIGS. 27, 29 and 31, respectively, schematically illustrate alternative embodiments of sheaths according to the present disclosure.

FIGS. 32 and 33 schematically illustrate a “tent” effect and how it may be mitigated.

FIGS. 34A and 35A, and their corresponding cross-sectional views shown in FIGS. 34B and 35B, respectively, schematically illustrate alternative embodiments of sheaths according to the present disclosure.

FIGS. 36 and 37 schematically illustrate a shearing effect and how it may be mitigated.

FIGS. 38-42 schematically illustrate various ways to mitigate puckering.

FIGS. 43-46 schematically illustrate sheaths according to alternative embodiments of the present disclosure utilizing a folded liner.

FIGS. 47A, 47B and 47C are perspective views of an introducer sheath system according to an alternative embodiment of the present disclosure.

FIG. 48A is a more detailed side view of the sheath shown in FIG. 47A, and FIGS. 48B and 48C are cross-sectional views taken along lines B-B and C-C, respectively, in FIG. 48A.

FIG. 49A is a more detailed side view of the dilator shown in FIG. 47A, and FIGS. 49C and 49B are cross-sectional views taken along lines C-C and B-B, respectively, in FIG. 49A.

FIG. 50A is a detailed side view of an alternative sheath for use in the system shown in FIG. 47A; FIGS. 50B and 50C are cross-sectional views taken along lines B-B and C-C, respectively, in FIG. 50A; and FIG. 50D is a detailed view taken at circle D in FIG. 50A.

FIGS. 51A-51C show detailed cross-sectional views of the sheath in the delivery or collapsed state (FIGS. 51A and 51A2); the expanded state (FIG. 51B); and the at least partially recovered state (FIGS. 51C and 51C2).

FIGS. 52A-52C are schematic partial top views showing various embodiments of openings in the liner for fluid delivery.

FIG. 53 is a longitudinal cross-sectional view of a portion of the sheath including the sheath hub.

FIGS. 54A-54C are perspective, end and side views of an example hemostasis valve for use in the hub shown in FIG. 53.

FIG. 55A is a detailed side view of an alternative dilator for use in the system shown in FIG. 47A; FIGS. 55B and 55C are a detailed views taken at circles B and C, respectfully in FIG. 55A; FIGS. 55D and 55E are cross-sectional views taken along lines D-D and E-E, respectively, in FIG. 55A; and FIG. 55F is a detailed view taken at circle F in FIG. 55E.

FIG. 56A is a side view of the sheath shown in FIG. 50A including a sheath protector; and FIG. 56B is a cross-sectional view taken along line B-B in FIG. 56A.

FIGS. 57A and 58 illustrate manufacturing flow charts for the sheath and dilator, respectively; FIGS. 57C and 57F show perspective views of mandrels to facilitate manufacture; and FIGS. 57B, 57D, 57E, 57G and 57H show cross-sectional views at various steps of manufacture.

FIG. 59A is a perspective view of an expandable introducer sheath according to an alternative embodiment of the present disclosure; FIG. 59B is a detailed perspective view of the distal portion of the sheath shown in FIG. 59A; and FIGS. 59C and 59D show the sheath of FIG. 59A in cross-section.

FIG. 60A is a perspective view of an expandable introducer sheath according to another alternative embodiment of the present disclosure; and FIGS. 60B and 60C show the sheath of FIG. 60A in cross-section.

FIGS. 61-64C show schematic perspective views of various ribcages for use in the sheaths of FIGS. 59A and 60A.

FIGS. 65A-65G show another alternative embodiment of an expandable introducer sheath.

FIGS. 66A-66D show various perspective views of an expandable guide extension catheter according to an embodiment of the present disclosure that utilizes the expandable sheath concepts and features described herein.

FIGS. 67A-67D illustrate various views of a dilator for use with the expandable guide extension catheter of FIGS. 66A-66D.

FIG. 68A shows a perspective view of an alternative embodiment of a guide catheter extension; and FIGS. 68B-68G show alternative constructions in cross-section.

FIGS. 69A-72 are schematic illustrations of various alternative embodiments of guide catheter extensions.

While embodiments of the disclosure are amenable to various modifications and alternative forms, specifics thereof have been shown by way of example in the drawings and will be described in some detail. It should be understood, however, that the intention is not to limit the disclosure to the particular embodiments described. On the contrary, the intention is to cover all modifications, equivalents, and alternatives falling within the spirit and scope of the invention.

### DETAILED DESCRIPTION OF EMBODIMENTS

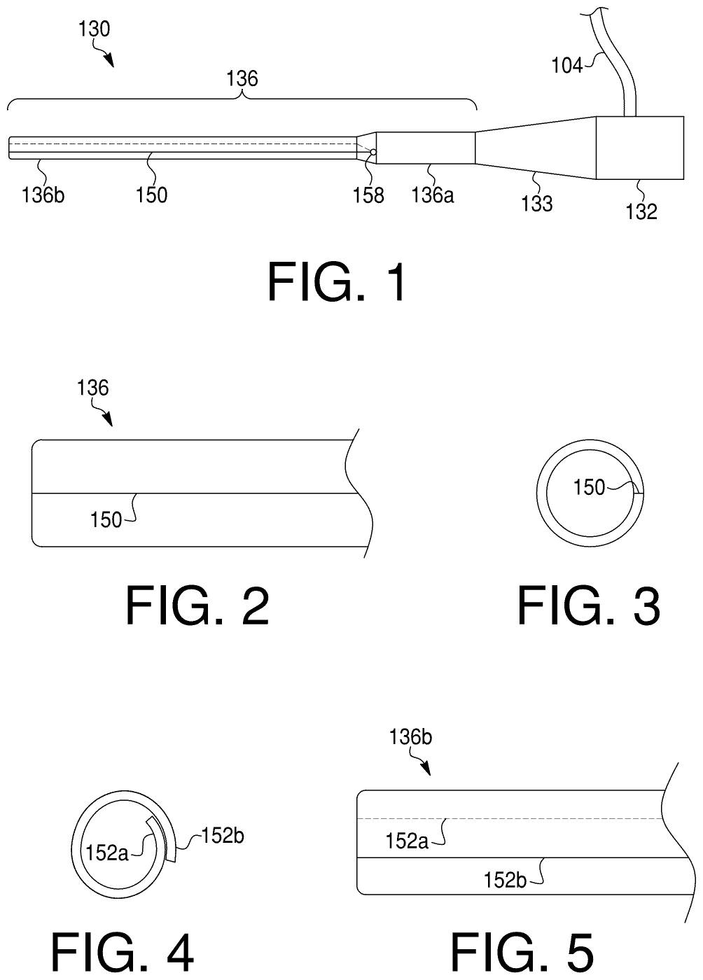

In one embodiment, shown in FIG. 1, an expandable sheath 130 is shown. Sheath 130 may include a tubular shaft 136, a hub 132, a flush line 104, and a strain relief 133. Hub 132 may contain a hemostatic valve. Shaft 136 may have a non-expandable portion 136A proximally, and an expandable portion 136B distally. Tubular shaft 136 may be longitudinally slit for a portion of the length of the shaft 136 and subsequently rolled or spiraled inwards, as will be described below.

FIGS. 2-5 illustrate steps for forming an expandable portion 136B of an embodiment of the present disclosure. A portion of the tubular shaft 136 is first longitudinally slit as shown in FIG. 2. This may be performed by cutting with a blade or other suitable means. Slit 150 is shown. FIG. 3 is a cross section of FIG. 2. Slit 150 extends through the wall. The slit 150 creates two flaps 152A and 152B.

Flaps may then be longitudinally tucked, one under the other similar to a spiral roll in cross-section, to create an overlap, as shown in FIG. 4, which is a cross section of FIG. 5. Dotted line indicates the end of the inner flap 152A.

The shaft portion 136B may be heat set or otherwise treated to naturally maintain this spiral overlapped condition. By being in a spiral condition, the outer diameter is therefore smaller than the pre-spiral condition. For example, a tubular shaft before slitting and spiraling may have an approximate inner diameter of 6 F. But after slitting, spiraling and heat setting it may have an approximate average inner diameter of 4 F or 5 F.

In one embodiment, the flaps 152A, 152B of the slitted sheath are spiraled down, reducing the outer diameter about 2 F and placing a setting tube 154 over the top as seen in FIG. 8. The spiraled sheath is then exposed to a warm temperature, such as approximately 225 to 275 degrees Fahrenheit to firmly set the reduced diameter. The setting tube 154 may then be removed, or left on the sheath and removed by the end user prior to use of the sheath in a blood vessel.

In one embodiment with a sheath 130 that is slit and spiraled, it may be introduced into a patient in a reduced diameter state. Once in position, typically a guide catheter or diagnostic catheter is introduced into the sheath. The body of these catheters is large enough to expand the sheath outward in a radial manner when advanced down the lumen of the shaft 136. Depending on the size of these catheters, the sheath may be expanded to nearly its original unspiraled size, either slightly less, the same, or even slightly larger than in the unspiraled condition, as in FIGS. 2 and 3.

Upon retraction of the internal catheters (guide catheters, diagnostic catheters and the like), the expandable sheath 130 may retract to a smaller diameter. Such reduction in diameter may be beneficial to preventing spasm, or mitigating the effects of spasm. In one embodiment, the sheath is able to “re-spiral” towards its initial spiraled condition, such as shown in FIGS. 4 and 5.

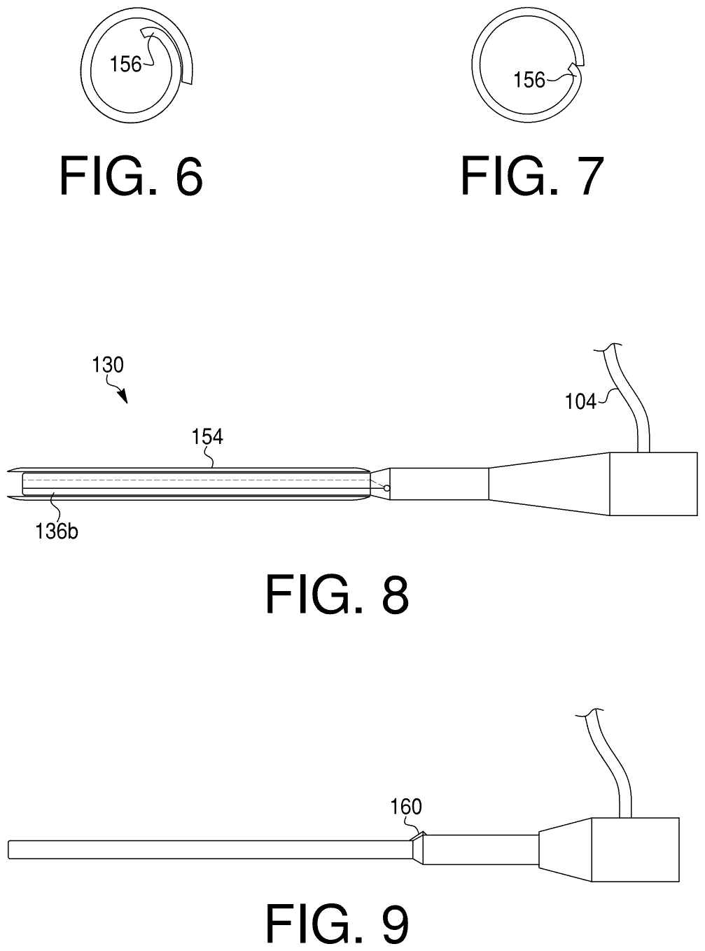

In one embodiment, one edge 156 of a slit flap 152A is further curled inward to provide a bias for the orientation of the slit edges upon retraction. Once expanded (by subsequent internal device placements), the curled edge 156 retains a surface inwardly of the non curled edge or flap 152B, as seen in FIGS. 6 and 7 (cross sections of unspiraled and spiraled sheath shafts 136, 136B), thus preventing any chance of the flaps “crossing over”.

In one embodiment, the longitudinal slit 150 with the flaps 152A, 152B (solid line and dotted line) are shown extending proximally to a point distal of the strain relief 133. The unslit portion of the tubular shaft may be unexpandable 136A and full diameter, for example with an ID of approximately 6 F, while the slit and reduced portion 136B is smaller diameter, for example approximately 4 F inner diameter. When introduced into the radial artery, in one embodiment, as shown here, the full diameter portion 136A would be introduced and reside in the arteriotomy, while the reduced diameter portion 136B would extend up the blood vessel such as a radial artery. Hemostasis at the arteriotomy is provided by this unslit portion. In an embodiment of the present disclosure, a small relief hole 158 is provided at the proximal end of the slit to avoid an outwardly projecting “pucker” or “tent” where the slit edges are adjoined. This relief hole 158 may have any shape, such as a circular cut as shown, or could be triangular, with the wider portion at the proximal end, or the distal end.

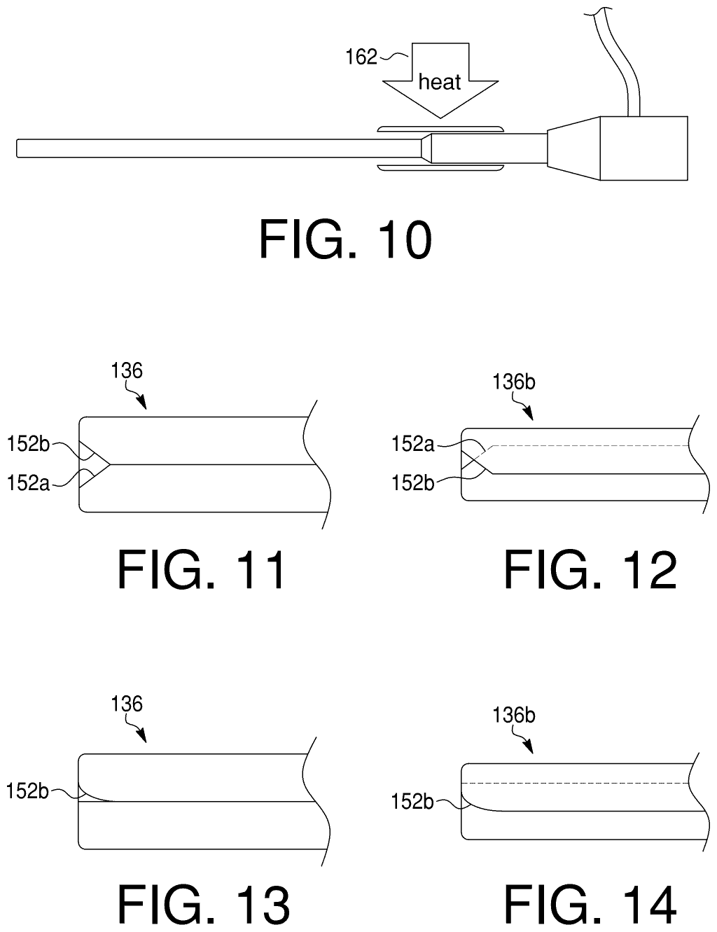

Alternatively, if a “pucker” or “tent” 160 is formed when the slitted sheath 130 is formed into a spiraled condition (as seen in FIG. 9), the tent may be thermally reflowed 162 back to a uniform outer diameter as shown in FIG. 10.

The slit portion 136B may allow for some fluid excursion along the length of the shaft. In cases where spasm does occur, the slit may provide the ability for some medications such as antispasmodics to be infused along the length of the sheath 130 and provide direct exposure of the vascular tissue to the medication.

In one embodiment, as seen in FIG. 11, the distal end edges of the flaps 152A, 152B are angled or curved. This helps to minimize the sharpness of the otherwise 90 degree end of the flaps.

When spiraled and collapsed down (FIG. 12), this embodiment may result in a distal end where the flaps 152A, 152B do not overlap each other, as seen in FIG. 12. This may aid in the distal tip having a lower profile when positioned over a dilator used together for introduction (not shown here).

In one embodiment, only one flap 152B has a rounded distal end, as seen in FIG. 13. Once the shaft portion 136B is spiraled and set (FIG. 14), the flap 152B resides outside of flap 152A. The rounded or angled edge of flap 152B serves to minimize “catching” or “hanging up” on tissue during introduction into a blood vessel, since there is no otherwise 90-degree edge corner.

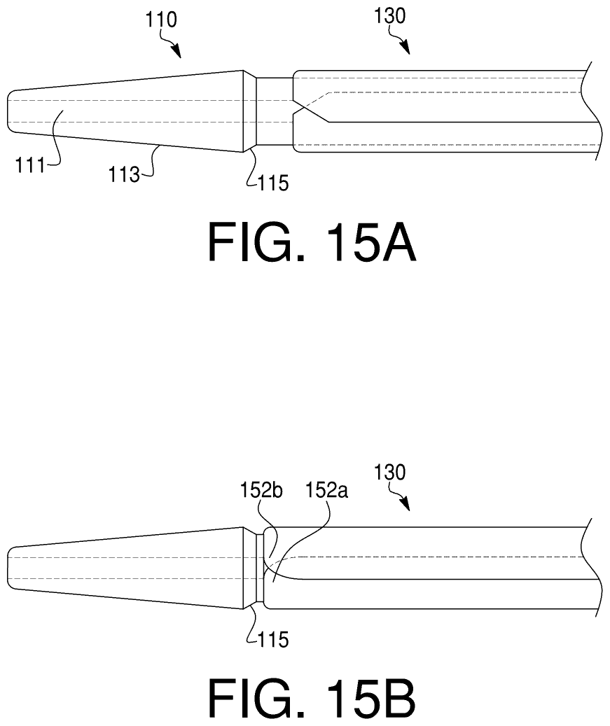

In one embodiment, expandable sheath 130 may be placed over a dilator 110 prior to insertion into a blood vessel such as a radial artery, as shown in FIG. 15A. The dilator 110 may have a wire lumen 111, for advancement over a pre-placed guide wire into the blood vessel. The dilator 110 may have a bulbous tip 113, with a reverse ramp 115 or transition that may be abutted to the distal end of the spiraled sheath. For illustration purposes, the reverse ramp 115 is shown extended somewhat distally. The reverse ramp 115 also serves to “shield” the distal end of the sheath from direct mechanical engagement with the arteriotomy (vascular access hole), minimizing trauma to this opening. Additionally, the dilator can have a shipping position and a delivery position. The shipping position may be somewhat distal, as shown. A reduction cover (not shown) can be present over the spiraled sheath, helping to keep it in its reduced diameter configuration. Prior to use, the cover may be removed by sliding it distally off the sheath 130 and over the bulbous tip 113 of the introducer. The dilator 110 may then be retracted until the reverse ramp 115 is just engaged or retracted slightly further to cause slight dilation and a press fit of the dilator 110 within the distal end of the spiraled sheath 130. The two positions of the dilator may be established by a two-position detent of the proximal hub of the dilator mating with the proximal end of the hub of the sheath (not shown).

FIG. 15B illustrates an embodiment where both of the flaps 152A, 152B have rounded distal ends. Having both flaps 152A, 152B rounded may facilitate retraction of the bulbous tip 113 proximally when it is desired to remove the dilator 110. Also, having both flaps 152A, 152B rounded may facilitate other devices that may be subsequently withdrawn into the sheath 130 and prevent those edges hanging up.

After the dilator 110 and sheath 130 are positioned in the blood vessel as desired, the dilator 110 can be removed proximally. The reverse ramp 115, working cooperatively with the ability of the sheath 130 to expand, allows for such a bulbous tipped dilator 110 to be utilized. As a system, this embodiment of dilator 110 and sheath 130 may be overall easier to introduce into the arteriotomy and blood vessel than conventional sheaths and dilator systems.

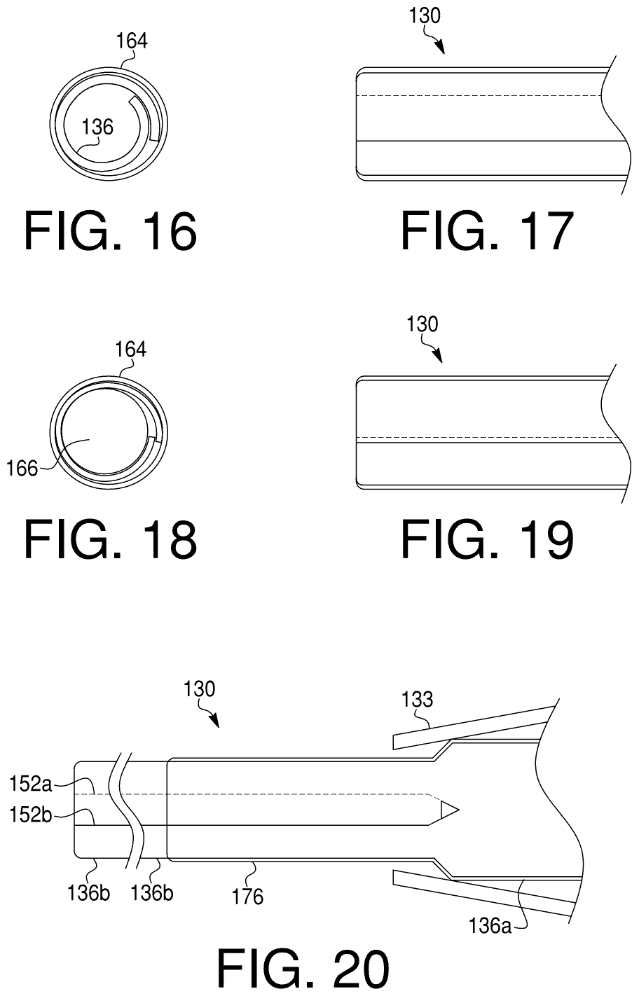

In one embodiment, seen in cross section in FIG. 16, and side view in FIG. 17, an elastomeric outer layer 164 is provided. This outer layer 164 may provide a seal to the slit 150 for containing potential fluids that may be injected, such as contrast fluid. The elastomeric outer layer may expand along with the spiraled sheath 130 in the presence of a catheter/device 166 such as a guide catheter or diagnostic catheter, shown in FIGS. 18 and 19. Upon removal of such a device 166, the elastomeric outer layer 164 further assists in reducing the diameter of the sheath 130 back towards its initial reduced diameter, shown in FIGS. 16 and 17.

The elastomeric jacket or outer layer 164 may be bonded to the outside of the spiraled sheath 130, or it may be unbonded. Or it may be partially bonded along one side of the sheath 130, and or at the distal end for some or all of the periphery or at other locations along the shaft 136 of the sheath 130. In an embodiment where the jacket is unbonded, it may be frictionally engaged with the outer surface of the sheath shaft 136. When the sheath 130 is expanded, the resulting strain in the elastomeric jacket or outer layer 164 may be distributed along nearly its full perimeter.

In one embodiment, an expandable sheath 130 may be formed wherein the entire length of the shaft 136 distal of the strain relief 133 may be slit, spiraled or otherwise reduced in diameter creating flaps 152A, 152B the extend the full length of the shaft 136, as can be seen in FIG. 20. Thus, the entire length of the sheath 130 that is introduced and advanced into a blood vessel, and within the arteriotomy, will be of reduced initial diameter 136B and subsequently expandable. A non-expandable larger diameter portion 136A may reside with the strain relief 133. In order to provide hemostatic sealing through the arteriotomy, a proximal elastic sleeve 176 may surround the proximal portion of the expandable shaft 136B

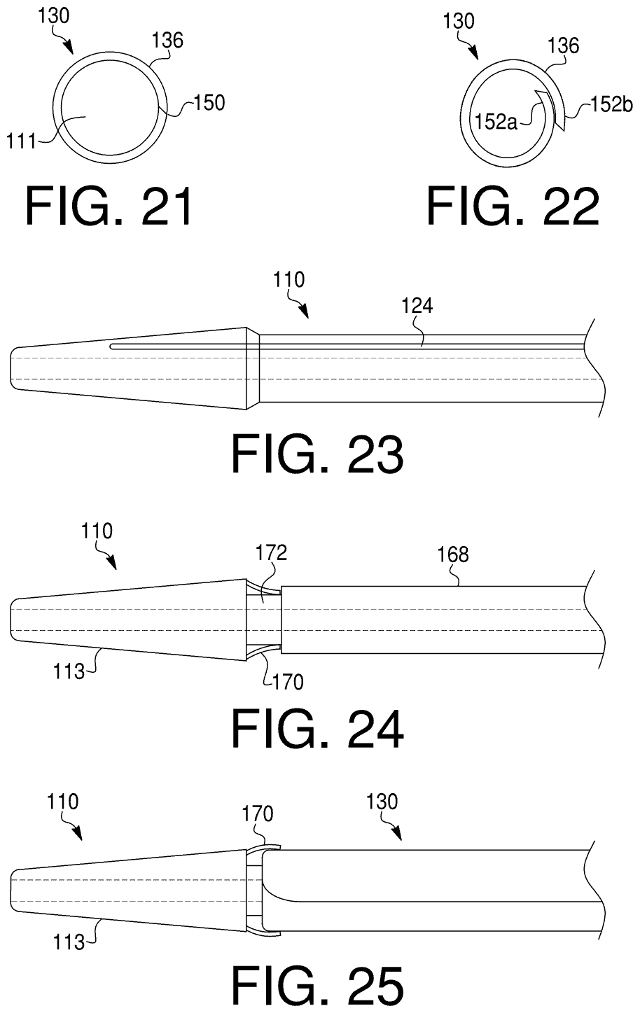

In one embodiment, the slit 150 that is created in the shaft 136 of the sheath 130 may be at an angle as shown in FIG. 21, and then set in the spiraled shape as seen in FIG. 22 with flaps 152A, 152B. When the sheath 130 is in use, after it is expanded by the luminal passage of a device such as a guide catheter (not shown), it may approach the original shape shown in FIG. 21. Upon removal of the device from the lumen 111, the sheath 130 may elastically reduce back in diameter towards the spiraled configuration. The angled edges will urge one flap 152A to consistently reside on the inside of the other flap 152B, as shown in FIG. 22.

As the dilator 110 for a spiraled sheath 130 as described herein may be smaller than that for a conventional non-slitted non-expandable sheath, the flexibility will tend to be greater. It may be desirable to “add back” the stiffness of the dilator 110 to that used with the conventional sheath. One embodiment of the dilator 110 may include a stiffening structure, such as an embedded metallic wire 124 or the like as shown in FIG. 23. Other embodiments may utilize a braid to add additional stiffness. Alternatively, the dilator 110 could be fabricated of more rigid polymers than might otherwise be utilized, such as relatively rigid nylon, PEEK, high durometer PEBAX or other rigid polymeric materials.

As shown in FIGS. 24 and 25, the dilator 110 may include multiple portions, such as a bulbous distal tip 113, a longitudinal body portion, and a recessed portion therebetween. Additionally, an elastomeric sleeve portion 170 (shown in longitudinal cross section) may be disposed proximal of the bulbous tip 113 (FIG. 24 in section).

When the dilator 110 and sheath 130 are loaded together, the elastomeric sleeve portion 170 is positioned about the distal end of the spiraled sheath 130, which overlaps and covers this portion of the sheath. This configuration may assist in smooth passage of the sheath/dilator into the blood vessel. Once the sheath 130 is positioned as desired, the dilator 110 may be advanced, which will allow the elastomeric sleeve portion 170 to retract back to the condition as shown in FIG. 24. Then dilator 110 may be withdrawn back into the sheath 130, presenting a smooth reverse tapered surface to the inside of the spiraled sheath 130.

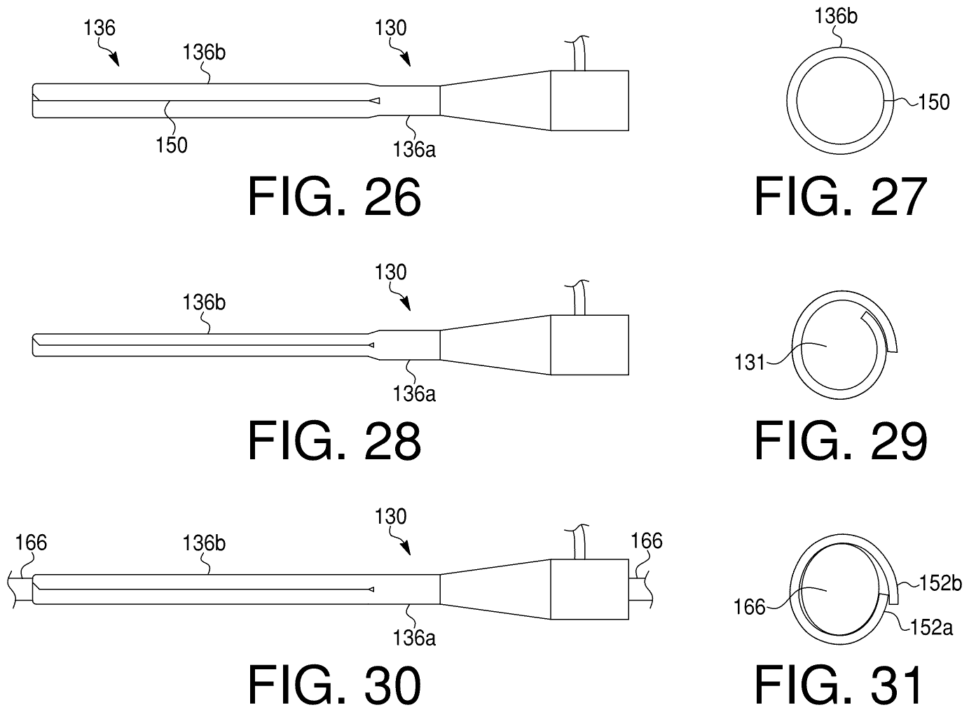

In one embodiment, the initial sheath shaft 136 may be formed from two diameters 136B, 136A, as shown in FIG. 26. The proximal diameter 136A is smaller, and of the desired size for the vascular access hole, i.e. arteriotomy or venotomy. For example, a common sheath size for radial access is 6 F, so in this example, the smaller diameter portion 136A of the sheath shaft 136 is 6 F (typically an ID measurement). The distal portion 136B may be initially larger, for example 7F or 8 F before the slit is formed. The slit 150 is formed only in the larger diameter portion 136B as shown. FIG. 27 is a cross section of the slitted portion 136B prior to being spiraled.

When the slitted portion 136B of the sheath shaft 136 is spiraled, the larger diameter portion 136B becomes smaller than the proximal portion 136A, as seen in FIG. 28. Thus, in this example the portion 136B to be introduced and advanced into the vessel has a smaller diameter of approximately 4 F or 5 F. FIG. 29 is the cross section of this distal portion 136B after being spiraled down.

When a guide catheter 166 or the like is advanced into the lumen 131, the distal sheath diameter 136B naturally expands, but only to approximately the same dimension as the proximal portion 136A of the sheath 136, as seen in FIG. 30. Sticking with the example, this would be approximately 6 F. Because the initial diameter of the distal sheath 136B was larger initially (FIG. 26), there will remain an overlap of the flaps 152A, 152B, as seen in the cross section FIG. 31. Because of this overlap, when the guide catheter 166 is removed, the flaps 152A, 152B of the slitted portion 136B of the are guaranteed to not “cross over” when they reduce back down towards the size shown in FIGS. 28 and 29.

In one embodiment, as seen in FIG. 8, the sheath 130 is slit longitudinally, resulting in two flaps 152A, 152B. The flaps 152A, 152B are spiraled, resulting in a reduced diameter 136B. An outer process tube 154 may create and maintain this reduced diameter condition. The sheath may then be “heat set” at a temperature which encourages the spiraled sheath 130 to maintain this smaller diameter 136B thereafter, after the process tube 154 is removed. This shape setting also provides the sheath 130 with a “memory” of its reduced configuration, thus enabling the sheath 130 to at least partially re-constrict after expansion in use from the presence of a catheter 166 dilating its interior lumen 131.

In one embodiment, the outer surface of the sheath 130 may be coated with a lubricious coating, such as a hydrophilic coating. The coating may be performed prior to the spiraling, diameter reducing process. Alternatively, the coating may be performed after the sheath 130 has been spiraled and its diameter reduced. In this embodiment, the outer surface of the sheath flap 152A that rests inside the adjacent flap 152B will not be exposed to the coating, and may remain uncoated.

In one embodiment, the dilator 110 may be coated with a lubricious coating. This coating may extend for part or all of the length of the dilator 110. In one embodiment only the exposed distal end of the dilator is coated.

In one embodiment, the cut window or relief hole 158 at the proximal end of the slit 150 may be used for injection of fluids, such as saline or pharmacological agents such as vasodilators. Injection via this window may expose the vessel wall outside of the sheath to these fluids. In one embodiment, injection of fluids through this port may be used to relubricate a hydrophilic coating on the sheath. In another embodiment, fluids may be injected and exit the lumen 131 via the space between the overlapping flaps 152A, 152B.

In one embodiment, the expandable sheath 130 is formed by slitting and spiraling down at least a portion 136B to enable it to be expandable. At the distal end of the sheath 130, a temporary “breakaway bond” 182 may be formed by thermal bonding, as shown in FIG. 46. Such a breakaway bond 182 may be formed over a relatively short length, or can be extended for some or all of the length of the reduced diameter portion 136B of sheath 130.

In one embodiment, the inner diameter of the sheath 130 may be coated by a lubricious coating such as silicone or hydrophilic coating.

Materials for the shaft 136 of embodiments of the sheath include but are not limited to High Density polyethylene, PEBAX block copolymers, PTFE, FEP, or multi-layer constructions of PTFE with Pebax, FEP with Pebax. Other materials include thermoplastic polyurethane (TPU), ETFE, PEEK. Another material includes an outer layer of Pebax and an inner layer of Pebax loaded with particles of PTFE or other lubricious material. In the latter embodiment, the PTFE particles are incorporated within the Pebax, and serve to present a lubricious inner surface for devices which pass into the lumen of the sheath.

In one embodiment, the spiraled sheath may be heat set by spiraling the flaps (such as shown in FIGS. 6 and 22) and placing a setting tube 154 on the outside, keeping the flaps 152A, 152B in a reduced diameter configuration. Polymers may take a set, or “creep” in the spiraled configuration, even at temperatures close to room temperature. When the setting tube 154 is removed by the user, the spiraled configuration 136B will remain, allowing for the spiraled sheath 130 to be in a low profile and flexible condition for introduction and advancement in the blood vessel. This imparting of a reduced diameter spiraled shape into the sheath may be referred to as “cold setting”.

Once in the body, the internal stresses preserving the spiraled shape and size 136B may largely relax such that when a catheter such as a guide catheter 166 are introduced into the sheath 130, the spirals will easily yield and expand to the larger diameter imparted by the catheter 166, and present relatively little friction between the flaps 152A, 152B of the sheath 130 and the catheter 166, both for longitudinal movement as well as rotational movement.

Certain materials suitable for the shaft 136 may yield and relax more readily, especially those with relatively low glass transition temperatures.

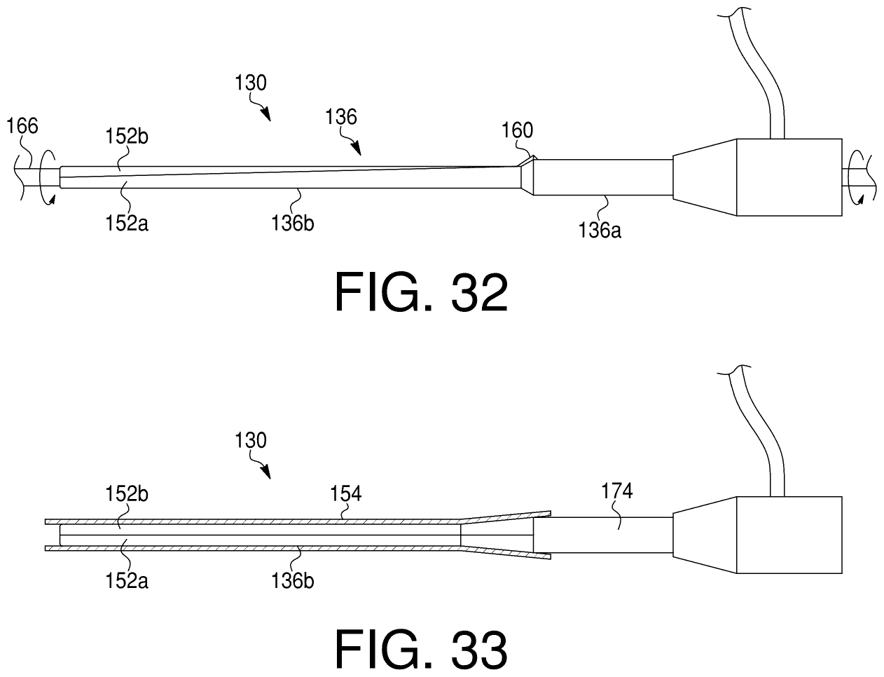

When the slitted spiraled sheath 130 described in the various embodiments is used with an enlarging catheter 166 such as a guide catheter, the enlarging catheter 166 will expand the inner diameter of the spiraled portion 136B of the sheath 130. Some degree of friction will naturally be present between the contacting flaps 152A, 152B and the catheter 166. This friction will create a longitudinal drag force on the catheter 166 during catheter advancement. If the catheter 166 is rotated within the sheath 130, the friction will serve to twist and shear the flaps 152A, 152B. The resulting twist, if great enough, can serve to create a “tent” 160 near the proximal end of the slit, shown in FIG. 32.

In one embodiment, to minimize or eliminate the “tent” due to twisting, a transition length 174 between the fully reduced spiral portion 136B and the unreduced portion 136A of the sheath 136 proximal of the slit portion with flaps 152A, 152B may be created. This transition length 174 may be established by incorporating the shape of transition length 174 into the setting tube 154, as shown in FIG. 33. The transition length 174 may be from about 1 mm to about 20 mm. The transition length 174 will serve to distribute torsional stresses over a longer length and minimize the formation of a tent 160.

In one embodiment, the tubular shaft 136 of the sheath may be rotationally coupled to the hub 132 and valve portion (not shown). In this embodiment, friction may arise between the inside of the sheath 130 and a catheter 166 such as a guide catheter enlarging the spiraled portion 136B of the sheath 130. When the catheter 166 is rotated, rather than the flaps 152A, 152B and slit twisting as a result of the torsional friction, the entire tubular sheath shaft 136 may rotate in response.

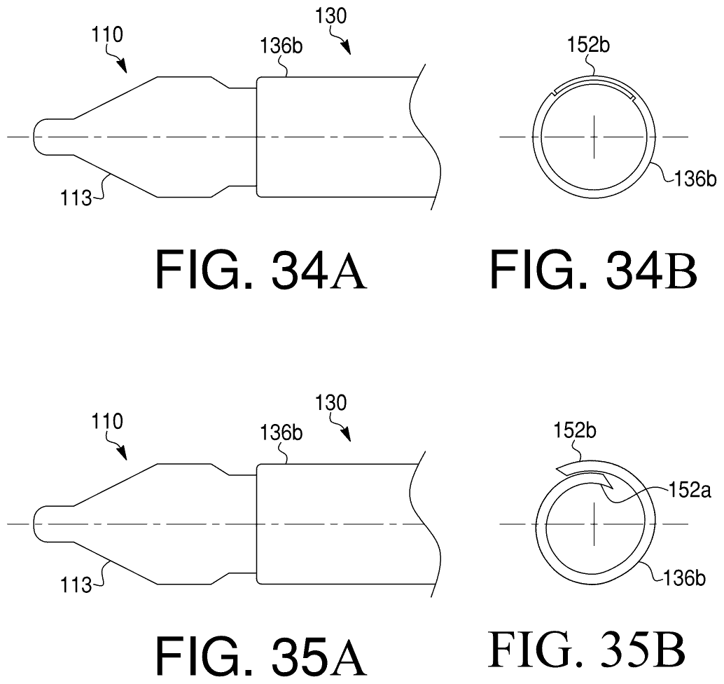

In one embodiment of an expandable sheath 130, the flaps 152A, 152B may be formed to be thinner in their overlapped regions when spiraled, as seen in FIG. 34B, a cross section of the distal portion 136B of the spiraled sheath 130. As seen in FIG. 34A, a dilator 110 with a bulbous tip 113 may therefore be concentrically formed for insertion together with the spiraled sheath 130, show here at the distal end of the sheath 130.

Alternatively, as shown in FIG. 35B, cross section of distal portion 136B of sheath 130 of FIG. 35A. If the flaps 152A, 152B are not formed to be thinner in their overlap region, the bulbous tip 113 of the dilator 110 may be eccentric as shown. In this manner, the dilator 110 serves to effectively “lead in” the thicker overlapped portions of sheath 130 into the blood vessel. The proximal end of the dilator 110 may be “keyed” into the hub 132 of the sheath 130 at its proximal end (not shown) to provide the proper relative rotational positions of the dilator 110 and overlapped portion of the sheath 130.

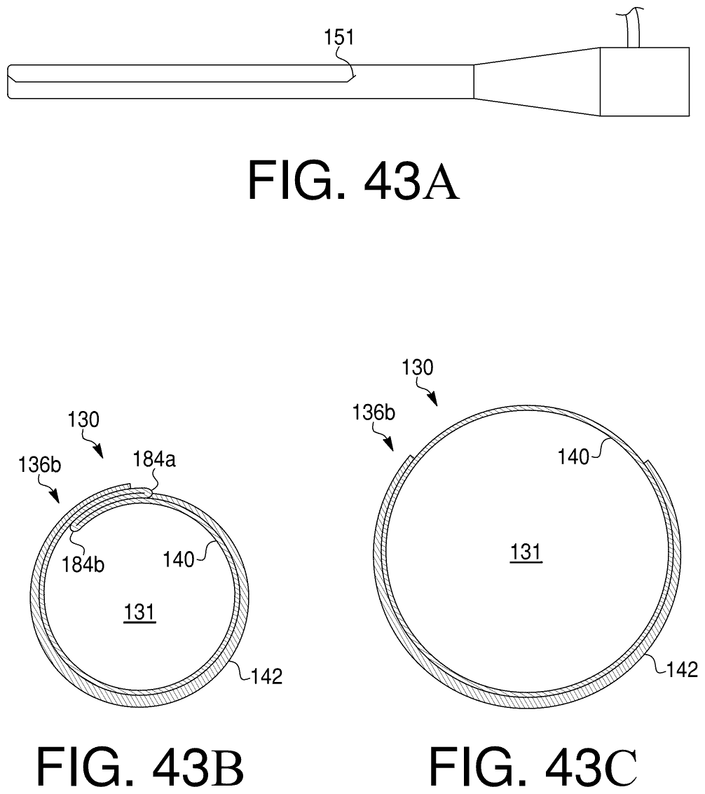

In one embodiment, the longitudinal slit 150 may terminate at its proximal end in a non-axial direction 151, as seen in FIG. 43A. The direction 151 may be offset by approximately 45 degrees, 90 degrees, or any suitable angle. It may be straight or curved. This non-axial length in the direction 151 of slit 150 may be less than approximately 1 mm or greater than approximately 1 mm. The non-axial length in the direction 151 of slit 150 may be longer for larger diameter sheaths. If the flaps 152A, 152B of the sheath 130 are twisted arising from torsional friction, any deformity leading to a “tent” 160 may be dispersed and minimized.

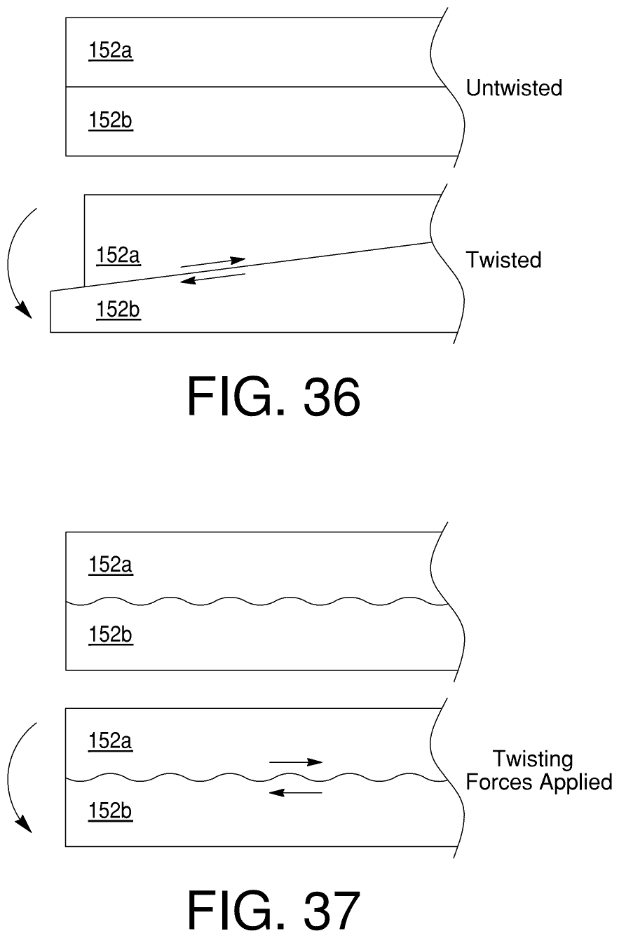

In the embodiments wherein the slit 150 is generally longitudinal, torsional friction that leads to twisting of the sheath body will tend to slide the flaps 152A, 152B in a shearing motion adjacent their edges, as shown in FIG. 36, showing distal portion of the sheath 130 in untwisted and twisted conditions. This shearing motion can lead to the formation of a tent or pucker 160, typically near the proximal ends of the flaps 152A, 152B, at the proximal end of the slit 150.

In one embodiment, when the expandable portion 136B of the sheath 130 is in its expanded condition, the longitudinal slit 150 at the edges of the flaps 152A, 152B has portions that are not straight, as shown in FIG. 37. These portions can be any non-straight shape, such as zig-zags, sine waves, square waves, partial square waves, and the like. When the twisting forces are applied, the edges lock in to each other and serve to prevent the twisting motion from arising. The non-straight slit 150 may be formed by mechanical cutting, laser cutting, or other suitable means.

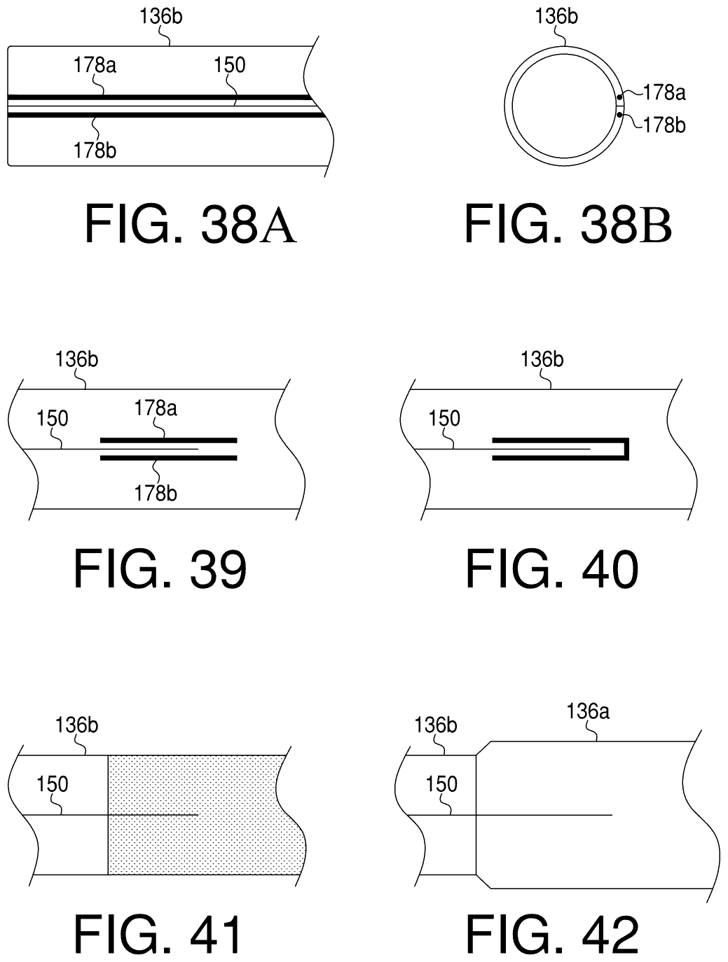

Another option for controlling tenting/puckering of the flap edges arising from torsional forces is to reinforce the stiffness of the flaps 152A, 152B near their edges. In FIG. 38A, longitudinal wires 178A, 178B may be of a relatively rigid material such as stainless steel are embedded near the edges of the flaps. FIG. 38B shows the cross-section of expandable portion 136B of sheath 130. The wires 178A, 178B may extend from the distal end of the sheath 130 to the proximal end adjacent the hub 132, or to a position proximal of the slit 150, but distal of the hub 132. Shown is the sheath 130 in the fully expanded condition. Alternatively, as shown in FIG. 39, the wires 178A, 178B may be present only near the proximal end of the slit 150, as this is where puckering is the most likely. Wires 178A, 178B may also be combined in a “U” shaped form wherein their proximal ends are joined, shown in FIG. 40.

As shown in FIG. 41, as an alternative way to reinforce the most susceptible portion of the sheath 130 and assist in preventing or minimizing puckering, the portion of the sheath near or at the proximal end of the slit 150 may be formed of a more rigid polymer than the portion distally. Sheath 130 here is shown in the expanded condition. By way of example, the main portion of the sheath may be formed from a particular Pebax durometer such as Pebax 72D. An adjacent portion just proximally and incorporating the proximal end of the slit 150 may be formed of a more rigid polymer such as Nylon. Alternatively, there may be several changes in material stiffness, from a point proximal of the proximal end of the slit and progressing to successively more flexible materials towards the distal end of the sheath.

As shown in FIG. 42, as another alternative way to reinforce the most susceptible portion of the sheath 130 and assist in preventing or minimizing puckering, a proximal portion of the sheath 136 including the proximal portion of the slit 150 may be formed of a thicker walled tube relative to the distal portion of the tube, as shown in FIG. 42. There may be a single change in wall thickness, as shown, or a more gradual change in wall thickness—from a point proximal of the slit 150 to a portion significantly more distal of the slit 150.

In an alternative embodiment of the present disclosure, the tendency for the slit 150 and flaps 152A, 152B to pucker due to torsional forces as described above may be reduced or eliminated by reducing the frictional forces between interior of the expanded sheath 130 and an indwelling catheter 166 (such as a guide catheter). Friction may be reduced by the incorporation of a lubricious interior liner such as PTFE. Furthermore, the coefficient of friction may be further reduced by incorporating a slightly roughened surface, similar to an “orange peel” surface.

Further reductions in the coefficient of friction on the interior of the sheath many be achieved with lubricious coatings such as slippery hydrophilic coatings.

An alternative way to reduce the frictional engagement of an indwelling catheter such as a guide catheter 166 and the expandable sheath 130 is to utilize materials that are known to “relax” at body temperature. Certain materials such as ethylene-tetrafluoroetylene (ETFE) are known to “creep” at body temperature. After an indwelling catheter 166 is introduced, and the expandable sheath 130 expands as a result, the frictional “grip” provided by the expanded sheath 130 will reduce over time if at body temperature. This reduction or relaxation may occur relatively quickly, such as within a few minutes or less. Once this relaxation occurs, the friction between the sheath 130 and indwelling catheter 166 will reduce, and subsequent torsional interactions are less likely to lead to puckering of the vulnerable portions of the sheath flaps 152A, 152B.

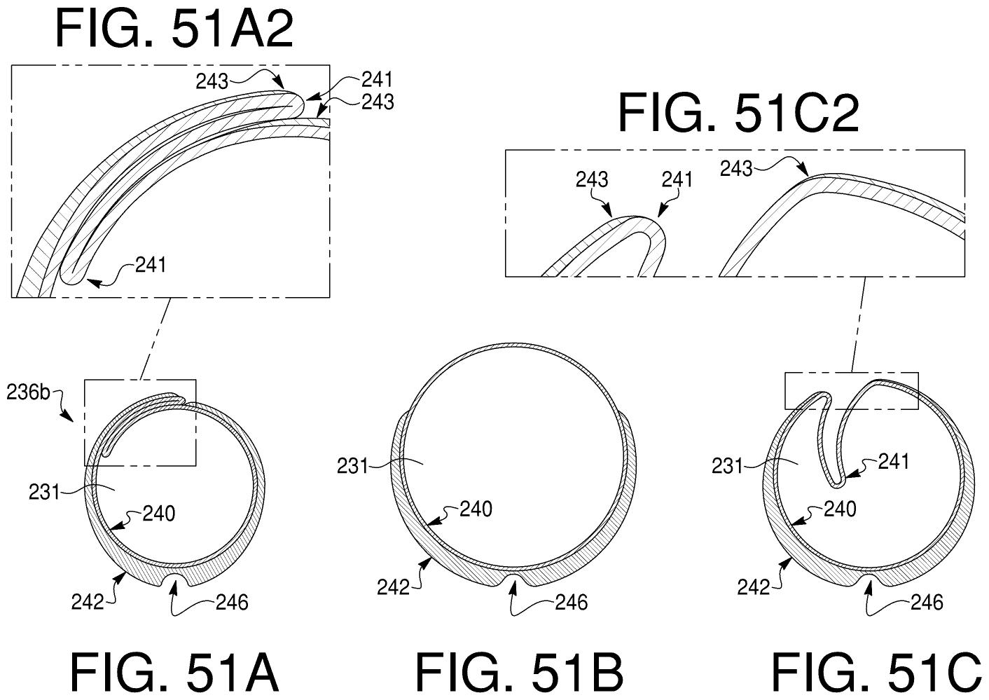

An alternative embodiment of the expandable sheath 130 is illustrated in FIGS. 43B and 51B. The sheath 130 may include two layers, an outer layer 142 with a nearly full circumferential perimeter (in the compressed reduced diameter condition as shown in FIG. 43B). An inner layer 140 is folded back on itself, forming two creases 184A and 184B. FIG. 43B shows the compressed, reduced diameter 136B of the sheath 130. Once expanded (for example, from the introduction of a larger diameter indwelling catheter 166 such as a guide catheter into lumen 131, the outer layer 142 becomes widened out as seen in FIG. 43C. The inner layer 140 unfolds and unfurls to a larger diameter to accommodate the larger indwelling catheter 166, as seen.

One possible advantage of this embodiment is that both sides of the outer layer 142 become cross-tied together. When exposed to torsional forces such as those arising from torsional friction between the inner surface of the sheath 130 and the outer surface of the indwelling catheter 166 when rotated, the sheath 166 will resist twisting and torsional puckering. This resistance to twisting may allow for relatively long expandable sheaths to be fabricated, e.g. lengths of approximately 10 cm or longer could be fabricated and still resist torsional puckering. For example sheaths 130 with lengths of approximately 15 cm, 20 cm, or longer, or in between may be made with this construction.

Suitable materials for this embodiment may include polymers such as Nylon, Pebax, PTFE, ETFE, HDPE, thermoplastic elastomers, and combinations of these materials.

In one embodiment, the sheath 136 may be fabricated from a single layer (as opposed to two layers) of composite material, (yet possess a profile similar to the composite profile shown in FIG. 43C), such as Pebax or Nylon filled with particles of a lubricious material such as PTFE. The tube may have an eccentric wall thickness, with a thin side and thick side. The thin side may be the portion of the sheath that is in-folded, as like the inner layer of FIG. 43B.

In another embodiment of the present disclosure, the sheath 130 may be two layers, as illustrated. The inner layer 140 may be PTFE, presenting a lubricious inner surface. The outer surface of the inner layer 140 may be etched to enhance the bond between it and the outer layer 142. The outer layer 142 may be Nylon or Pebax or other suitable material. The reduced diameter condition 136B shown in FIG. 43B may be “heat set”. Heat setting provides for the re-folding, at least partially, after the larger diameter indwelling catheter 166 is removed from the lumen 131, towards a condition approaching that of FIG. 43B.

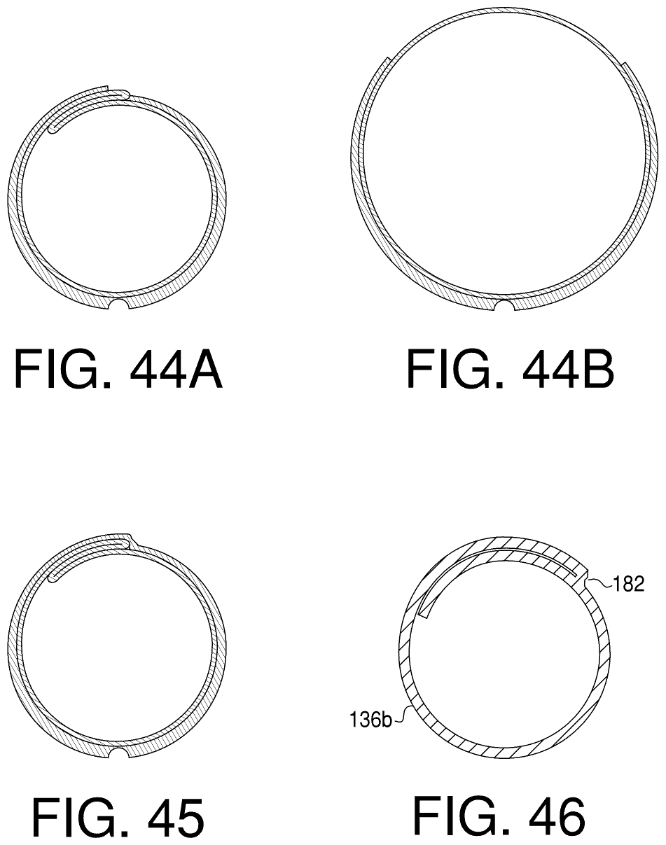

This embodiments of FIGS. 43B and 43C may be formed by a process of “over extrusion”, where the inner layer is drawn through an extruder that applies the outer layer to the inner layer in the profile. This over extrusion may then be formed into the sheath, whereby most or all of the length of the sheath is formed into the reduced diameter as seen in FIG. 44A. A proximal portion of the sheath may be unreduced, similar to embodiments described above.

In one embodiment, as shown in FIGS. 43B and 43C, the outer layer may be thinner at the edges (top, in figure).

Portions of the inner layer may furthermore include perforations in order to facilitate fluid delivery through the wall of the sheath.

The embodiments described in connection with FIGS. 43B and 43C may further include a “hinge”, as shown in FIGS. 44A and 44B. The hinge may be a longitudinal notch either partially or fully through the outer layer, as seen. The hinge allows for the sheath to radially expand to the condition seen in FIG. 44B with less dilating force. The frictional forces between the sheath and the indwelling catheter will subsequently be minimized in comparison to a non-hinged embodiment.

Furthermore, the presence of a hinge slows the speed of recover of the sheath to a reduced diameter after the indwelling catheter is removed. Slowing this recovery allows more time for the inner layer to properly refold, which may aid in the reduction of overall diameter of the sheath after the indwelling catheter is removed.

A hinge may also be provided in an embodiment wherein this embodiment is formed of a single layer with eccentric wall thickness.

Any of the expandable sheath embodiments described above can further include a seal or bond adjacent the distal end. Such a bond stabilizes the distal end of the reduced diameter sheath and prevent the flaps or in-folds from opening up during introduction into the tissue of the skin, artery wall, or other tissue as the sheath is being introduced into the lumen of the vessel it is being inserted in. The bond may be a “breakaway” in that the bond will break or release when the sheath is thereafter expanded by introduction of an indwelling catheter such as a guide catheter. Such a bond is shown in FIG. 46 for the spiraled embodiments, and in FIG. 45 for the in-folded embodiments. The bond may be formed by re-melting the material of the spiraled sheath (FIG. 46), or by remelting of the outer layer (FIG. 45) in the in-folded embodiments utilizing two layers. The bond may also be formed by introduction of an additional material such as an adhesive. The breakaway bond may be present for a relatively short distance adjacent the distal end of the reduced diameter portion of the sheath, e.g. for less than approximately 1 mm, or less than approximately 2 mm.

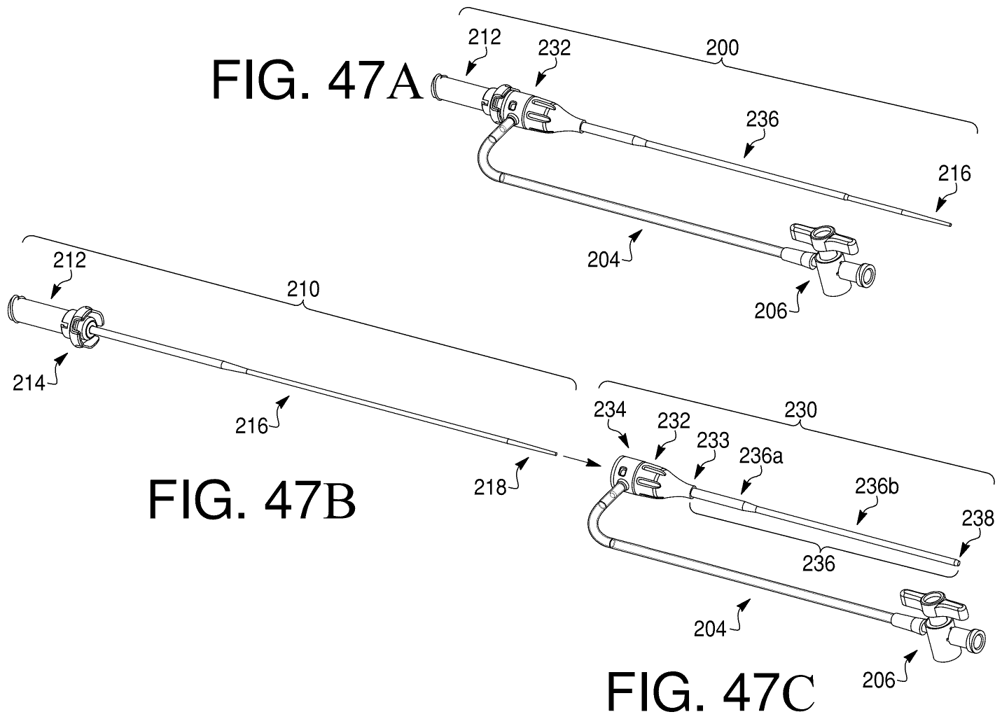

With reference to FIG. 47A, a perspective view of an expandable introducer sheath system 200 is shown. In this embodiment, the introducer sheath system 200 as shown and described may be used for small bore applications (less than approximately 8 F, for example). The introducer sheath system 200 may include two primary components, namely a dilator assembly 210 and a sheath assembly 230, as shown disassembled in FIGS. 47B and 47C, respectively. The introducer sheath system 200 may also include a flush line 204 with associated stop cock 206, which may be a one-way or a two-way stop cock, for example.

The dilator 210 may include a hub 212 having a snap cap 214. The hub 212 may be connected to a dilator shaft 216 having a multilayer construction, an outer diameter to match the unexpanded inside diameter of the sheath 230, and a length sufficient such that the tapered tip 218 extends beyond the tip of the sheath 230 when assembled. In particular, the dilator shaft 216 may have a proximal full diameter section or portion 216A having a diameter (e.g., approximately 6 F) corresponding to the inside diameter of the non-expandable portion or section 236A of the of the sheath shaft 236, a reduced diameter distal section or portion 216B having a diameter (e.g., approximately 4 F) corresponding to the inside diameter of the expandable portion or section 236B of the of the sheath shaft 236 when in a non-expanded state, a tapered diameter section 216C corresponding to the inside diameter of the transition portion or section 236C of the of the sheath shaft 236, and a tapered tip 218 that transitions down to the diameter of the guidewire (not shown).

The sheath 230 may include a hub 232 containing a hemostasis valve (not visible) and a snap cap 234 that releasably connects to the snap cap 214 of the dilator 210. The hub 232 may further include a strain relief 233 for connection to the sheath shaft 236. The sheath shaft 236 may include a non-expandable portion 236A and an expandable portion 236B. Optionally, the non-expendable portion 236A may include a proximal continuation of the expandable portion 236B with an elastic tubular cover. The non-expandable portion 236A (or the elastic tubular cover) may provide a uniform outer surface to enable effective hemostasis at the arteriotomy.

All or a portion of the sheath shaft 236 may be coated with a lubricious coating such as a hydrophilic coating (HPC). Optionally, the non-expandable portion 236A may remain uncoated to mitigate sheath 230 back-out and provide an area for the application of medical tape for securement.

The expandable portion 236B may include a longitudinal slit, wherein the edges of the slit may be connected to each other via a webbing. The webbing may include holes or otherwise be porous to accommodate the injection and infusion of liquids (e.g., saline or vasodilator solution, for example) between the sheath shaft 236 and the intima of the blood vessel into which the sheath 230 is inserted. This may be useful to improve hydration of the HPC, mitigate vessel spasm, etc.

To maintain the sheath shaft 236 in a collapsed configuration during insertion and to maintain a smooth transition between the dilator tip 218 and the distal tip portion 238 of the sheath shaft 236, the distal tip portion 238 of the sheath shaft 236 may be configured such that the edges of the slit are temporarily bonded in the area where they overlap. Such temporary bond may include a reflow of material over a short length, for example. The temporary bond may be broken upon insertion of a larger device (e.g., guide catheter) to expand the expandable portion 236B of the sheath shaft 236.

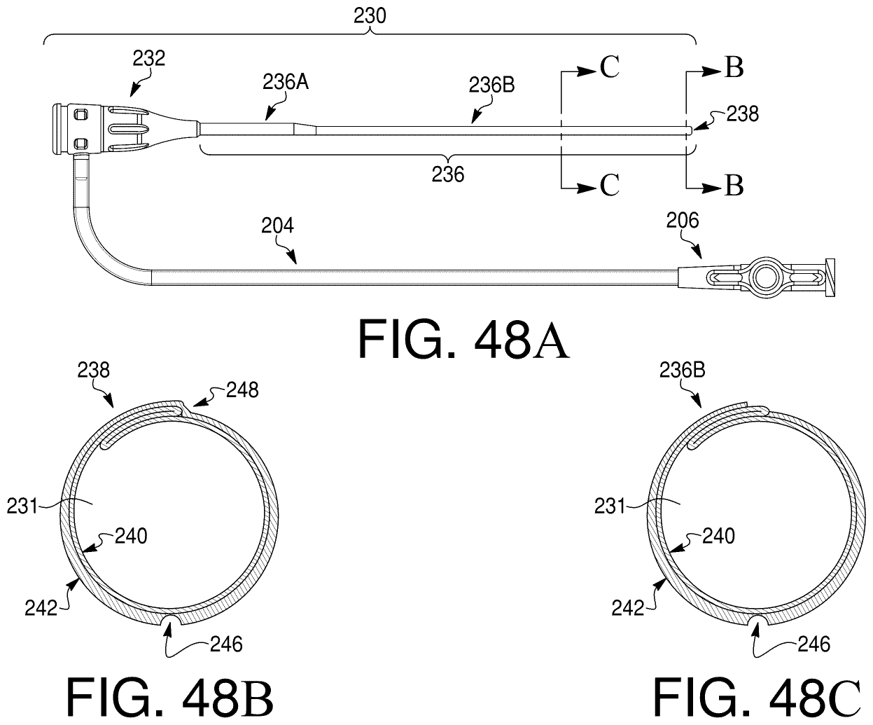

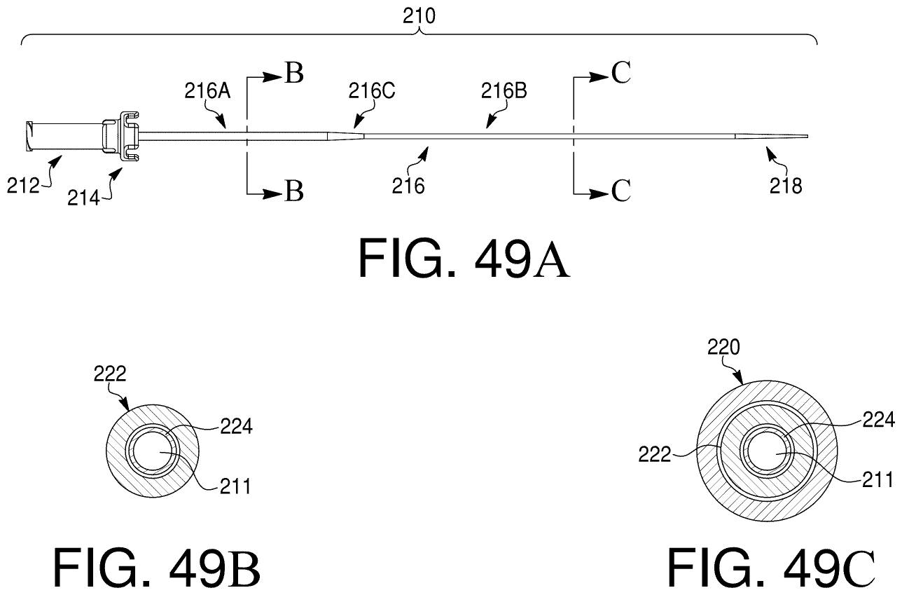

The dilator 210 may include a lumen 211 (visible in FIGS. 49B and 49C) to accommodate a guidewire (not shown). Similarly, the sheath 230 may include a lumen 231 (visible in FIGS. 48B and 48C) to accommodate the dilator 210. With this arrangement, the sheath assembly or system 200 may be advanced and withdrawn over a guidewire, and the dilator 210 may be advanced and withdrawn from the sheath 230. During advancement of the sheath assembly or system 200 over a guidewire, the dilator 210 may be releasably locked to the sheath 230 (via snap caps 214 and 234) and subsequently unlocked to withdraw the dilator 210 from the sheath.

With reference to FIG. 48A, a detailed top view of the sheath 230 is shown. FIG. 48C shows a cross-sectional view of the expandable portion 236B taken along line B-B in FIG. 48A, and FIG. 48B shows a cross-sectional view of the distal tip portion 238 taken along line B-B in FIG. 48A.

In general, the sheath shaft 236 may include an inner tubular liner 240 and an outer tubular jacket 242. The liner may be formed of a thin-walled lubricious material such as PTFE, and the jacket may be formed of a conventional catheter material such as poly-ether-block-amide (e.g., Pebax). The outer tubular jacket 242 may be slit along its length, whereas the liner 240 may be contiguous along its length, thus forming a membrane or webbing between the edges of the slit jacket 242 when expanded.

As seen in FIG. 48C, the outer jacket 242 may include a hinge 246 along its length to facilitate lower expansion force and to reduce the compressive forces once expanded. The hinge 246 may include, for example, a thinning of the outer jacket 242 wall thickness as shown.

As seen in FIG. 48B, the outer jacket 242 may have a temporary bond 248 such that the edges of the slit are temporarily bonded at the distal tip portion 238. Such temporary bond may include a reflow of the jacket 242, for example, thus temporarily closing the slit. The temporary bond 248 may extend proximally along the entire length of the expandable shaft 236B, or any distance proximally from the distal tip portion 238, continuously or intermittently.

With reference to FIG. 49A, a detailed top view of the dilator 210 is shown. FIG. 49C shows a cross-sectional view of the dilator shaft 216 corresponding the expandable portion 236B taken along line C-C in FIG. 49A, and FIG. 49B shows a cross-sectional view of the dilator shaft 216 corresponding the non-expandable portion 236A taken along line B-B in FIG. 49A.

In general, the dilator shaft 216 may include an outer jacket 220, an inner jacket 222 and a tubular stiffening member 224. As compared to conventional dilators that are monolithic, a composite construction may provide for different outside diameters corresponding to the inside diameters of the non-expandable section 236A and the expandable section 236B. Also, such composite construction may provide for different material selection to increase stiffness proximal of the tapered tip 218. This may off-set the loss in stiffness due to the reduced diameter (in the portion of the dilator shaft 216 corresponding to the expandable section 236B in the collapsed state). For example, whereas the outer jacket 220 and inner jacket 222 may include a conventional polymer such as LDPE or polyether-block-amide (e.g., Pebax), the stiffening member may include a stainless steel hypotube or a braid reinforced polymer, for example.

By way of example, n for an approximately 4 F to 6 F expandable sheath embodiment, the components may have the following characteristics in terms of dimensions and materials.

The sheath hub 232 may have a length of approximately 1.23 inches. The non-expandable portion 236A may have a length of approximately 0.79 inches. The transition between the non-expandable portion 236A and the expandable portion 236B may have a length of approximately 0.20 inches. The expandable portion 236B may have a length of approximately 3.15 inches. The non-expandable portion 236A may have a nominal inside diameter of approximately 0.086 inches and a nominal outside diameter of approximately 0.096 inches. The expandable shaft portion 236B may have a nominal unexpanded inside diameter of approximately 0.055 inches and may have a nominal unexpanded outer diameter of approximately 0.070 inches.

The dilator hub 212 may have a length of approximately 1.14 inches. The length of the dilator shaft 216 corresponding to the non-expandable portion 236A of the sheath shaft 236 may have a length of approximately 1.95 inches. The length of the dilator shaft 216 corresponding to the expandable portion 236B of the sheath may have a length of approximately 3.81 inches. The transition of the dilator shaft 216 between those sections may have a length of approximately 0.375 inches. The distal tapered tip 218 of the dilator 210 may have a length of approximately 0.75 inches.

With regard to the sheath 230, suitable materials for the hub may include Nylon, Pebax and HDPE. Suitable materials for the hub cap may include polypropylene and ABS. Suitable materials for the hemostasis valve may include silicone and polyurethane. Suitable materials for the strain relief include Pebax, polyurethane and TPE. Suitable materials for the jacket may include Pebax and nylon. Suitable materials for the liner may include PTFE or a blend of PTFE and Pebax. Suitable materials for the flush line may include PVC and polyurethane.

With regard to the dilator 210, suitable materials for the hub and snap cap may include nylon Pebax, HDPE and ABS. Suitable materials for the inner and outer jackets may include nylon, Pebax, PEEK and HDPE. Suitable materials for the stiffening member may include stainless steel hypotube with optional spiral cut, PEEK, LCP, and braid reinforced PI.

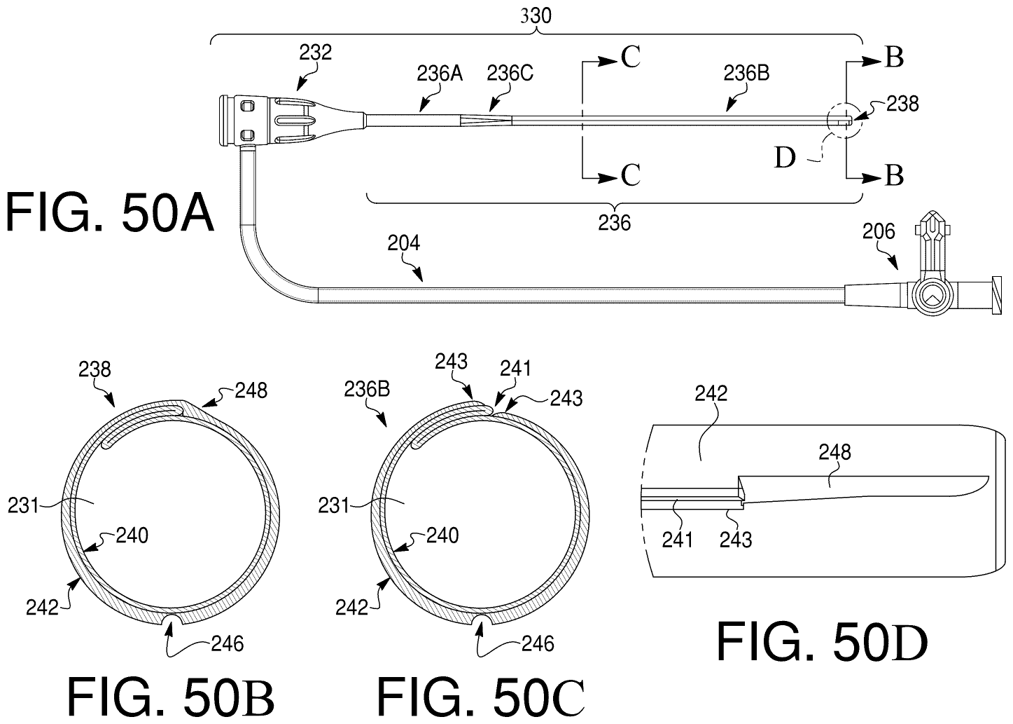

With reference to FIG. 50A, a perspective view of an alternative expandable sheath 330 is shown, which may be similar in many aspects to expandable introducer sheath 230 described previously. In general, common elements between introducer sheaths 230 and 330 are numbered the same. As compared to the prior embodiment, introducer sheath 330 may have a longer transition or tapered section 236C between the non-expandable section 236A and the expandable section 236B for smoother insertion into the skin and/or artery. As in the prior embodiment, but shown here in more detail, FIG. 50D shows the temporary bond 248 at the tip 238. The temporary bond 248 may include a thermal reflow forming a bridge between of the edges 243 of the outer jacket 242. Proximal of the bond 248, a fold 241 of the inner liner 240 may be seen in a gap between the edges 243 of the outer jacket.

FIG. 50B shows a cross-sectional view of the distal tip portion 238 taken along line B-B in FIG. 50A. FIG. 50C shows a cross-sectional view of the expandable portion 236B taken along line C-C in FIG. 50A.

As seen in FIG. 50B, the outer jacket 242 may have a temporary bond 248 such that the edges of the slit are temporarily bonded at the distal tip portion 238. Such temporary bond may include a reflow of the jacket 242, for example, thus temporarily closing the slit. The temporary bond 248 may extend proximally along the entire length of the expandable shaft 236B, or any distance proximally from the distal tip portion 238, continuously or intermittently

As shown in FIG. 50C, the outer jacket 242 may include a hinge 246 along its length to facilitate lower expansion force and to reduce the compressive forces once expanded. The hinge 246 may include, for example, a thinning of the outer jacket 242 wall thickness as shown.

FIGS. 51A-51C show a cross-sectional view taken along line B-B in FIG. 50A to illustrate the collapsed, expanded and semi-recovered configurations, respectively. As seen in FIG. 51A, where the expandable portion 236B is in a collapsed configuration for insertion into the skin/artery, and the detailed view in FIG. 51A2, the inner liner 240 may have two folds 241 facing inside toward the lumen 231 such that the folded inner liner 240 does not extend outside between the edges 243 of the outer jacket 242. Each of the folds 241 may be defined by two folded flaps of the inner liner 240, where the folds 241 share a common flap.

As seen in FIG. 51B, when the expandable portion 236B is in an expanded configuration, for example by inserting a guide catheter (or any other device that is larger than the collapsed lumen) into the lumen 231, the inner liner 240 may unfold, thus increasing the inside diameter of the lumen 231 to accommodate the guide catheter (or any other device). Simultaneously, the hinge 246 formed in the outer jacket 242 may open to accommodate expansion. When the guide catheter is removed, the expandable portion 236B may fully or partially recover as shown in FIG. 50D, with or without external compressive forces.

To enable full or partial recovery without external compressive forces, a number of features may be used to bias the folds 241 inward and close the expandable portion 236B of the sheath. For example, the outer jacket 242 at the hinge 246 may have a memory of its collapsed condition such that it is biased toward closure. Also, by way of example, the outer jacket 242 at the edges 243 that extend over the folds 241 may also have a memory of its collapsed condition such that it is biases the flaps inward. Further, the folds 241 may be configured to retain some memory of their folded shape such that they are biased toward a folded, collapsed configuration.

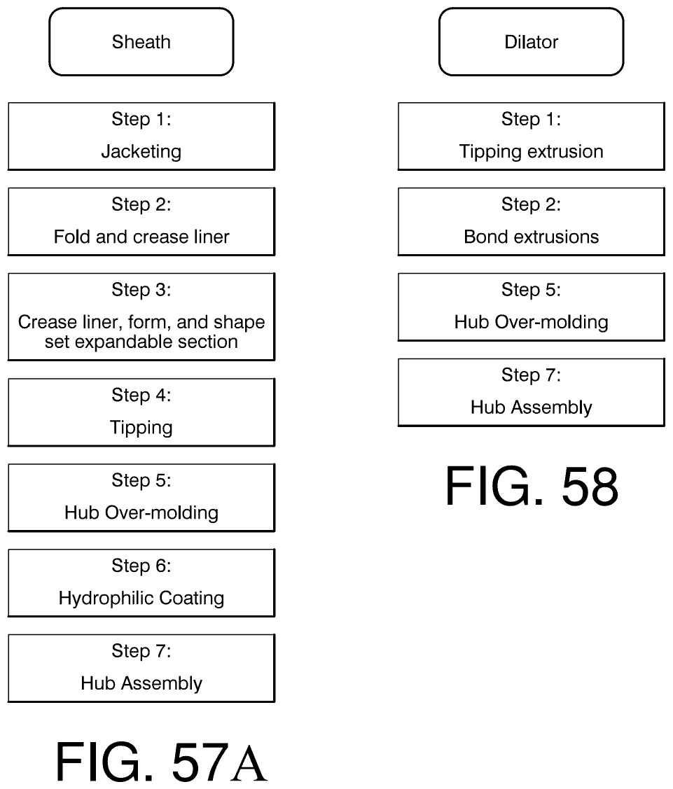

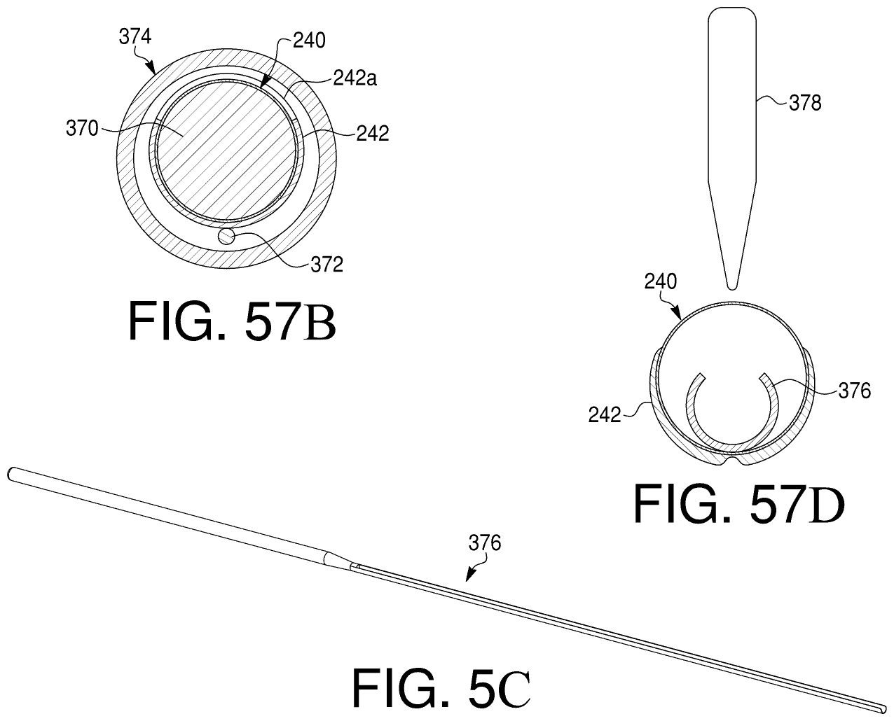

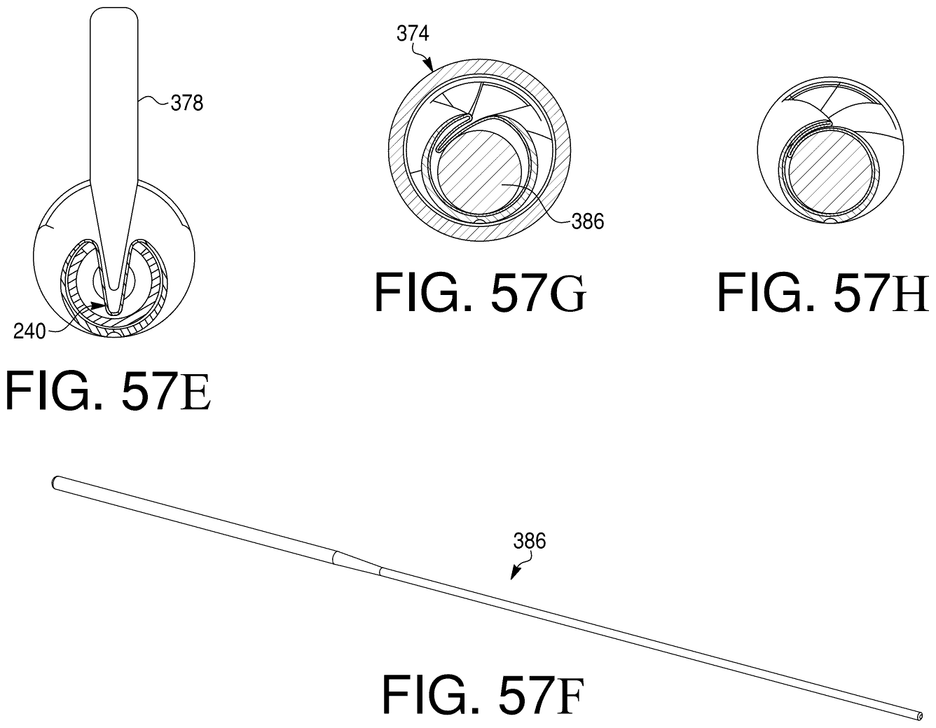

To achieve such memory in the folds of the inner, each fold 241 may include a crease. Such crease may include a plastically deformed inner 240 at the fold 241. Such crease may further include a crazed surface of the inner liner 240 on the outside of the folds 241. Such crazed outer surface of the liner 240 at the folds 241 may include microscopic gaps and fibrils in the material of the liner 240. Crazing has been described as a type of inelastic, plastic deformation that delays crack opening and allows the material to absorb more deformation energy before failure. The process described with reference to FIG. 57A may be employed to form the creased and/or crazed folds 241.

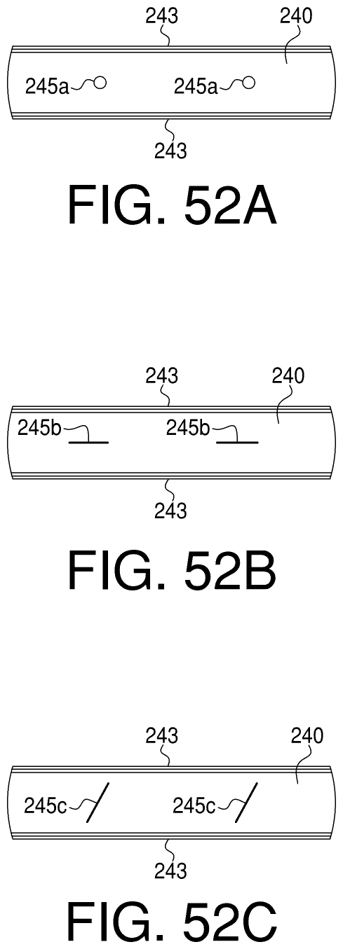

With reference to FIGS. 52A-52C, top views of the expandable sheath portion 236B are shown, including the unfolded liner 240 and the edges 243 of the jacket 242. As shown, the liner 240 may include holes 245A, longitudinal slits 245B or angular slits 245C or the like to accommodate the injection and infusion of liquids (e.g., saline or vasodilator solution, for example) between the sheath shaft 236 and the intima of the blood vessel into which the sheath 230 is inserted. This may be useful to improve hydration of the HPC, mitigate vessel spasm, etc.

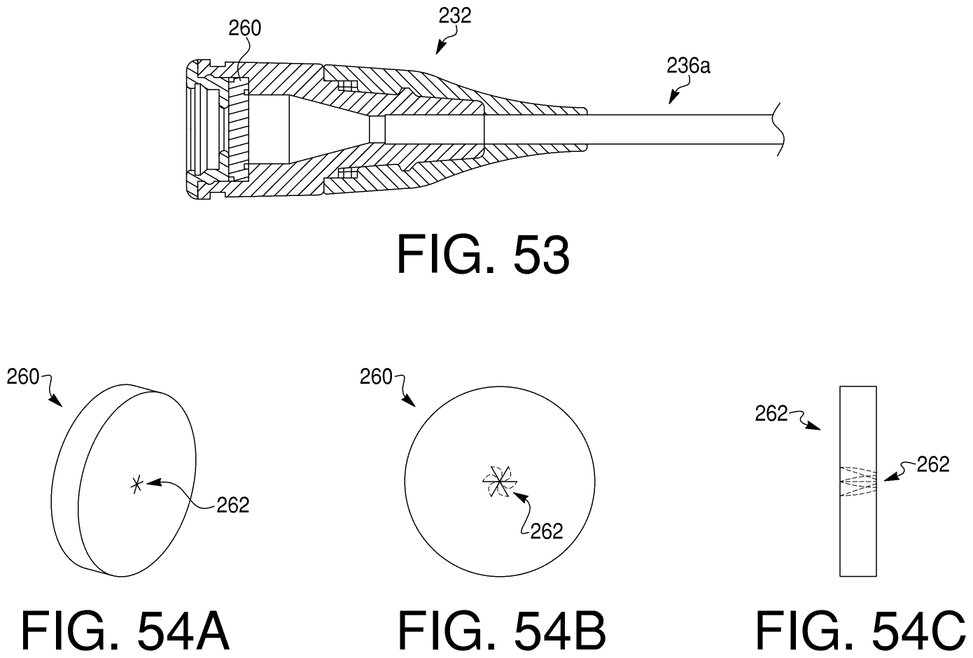

With reference to FIGS. 53 and 54A-54C, an example of a hemostatic valve 260 is shown. FIG. 53 shows the hub 232 in longitudinal cross section connected to the non-expandable sheath portion 236A. Within the hub 232, the hemostatic valve 260 may be secured between a cap and body defined by the hub 232. As shown in FIGS. 54A-54C, which are perspective, front and side views, respectively, the hemostatic valve 260 may include a conventional rubber or elastomeric gasket in a disc configuration, with a center slit 262 to accommodate the insertion of devices therethrough. In this example embodiment, the center slit 262 may include multiple slits distributed around a center axis as shown in FIG. 54A. As each of the slits extends through the gasket, the slit may be rotated about the center axis, resulting in a flower shape as shown in FIG. 54B and a crown shape as shown in FIG. 54C. This configuration may provide better hemostasis and/or lower friction.

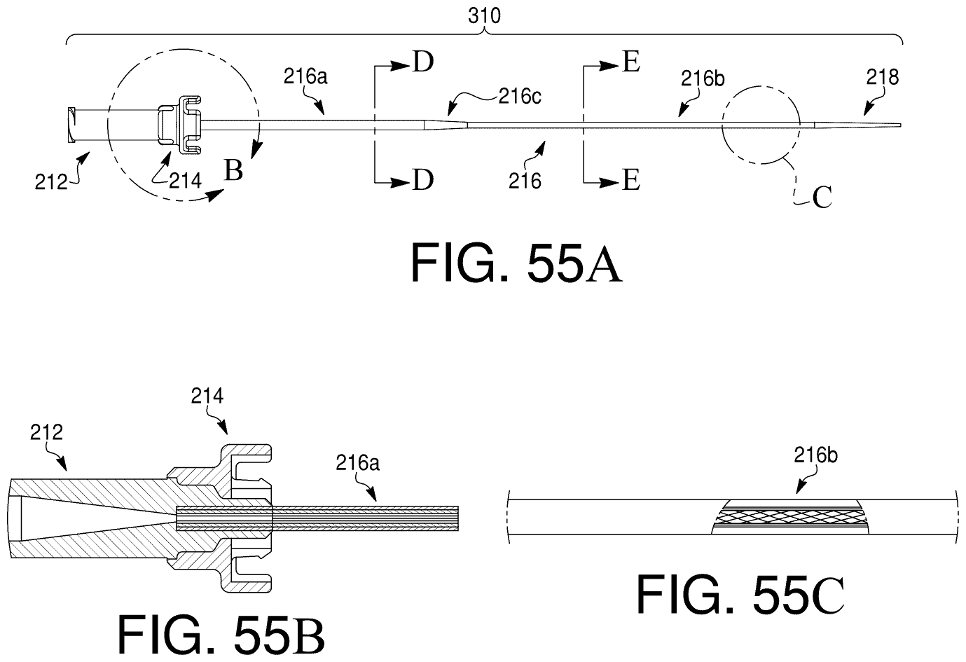

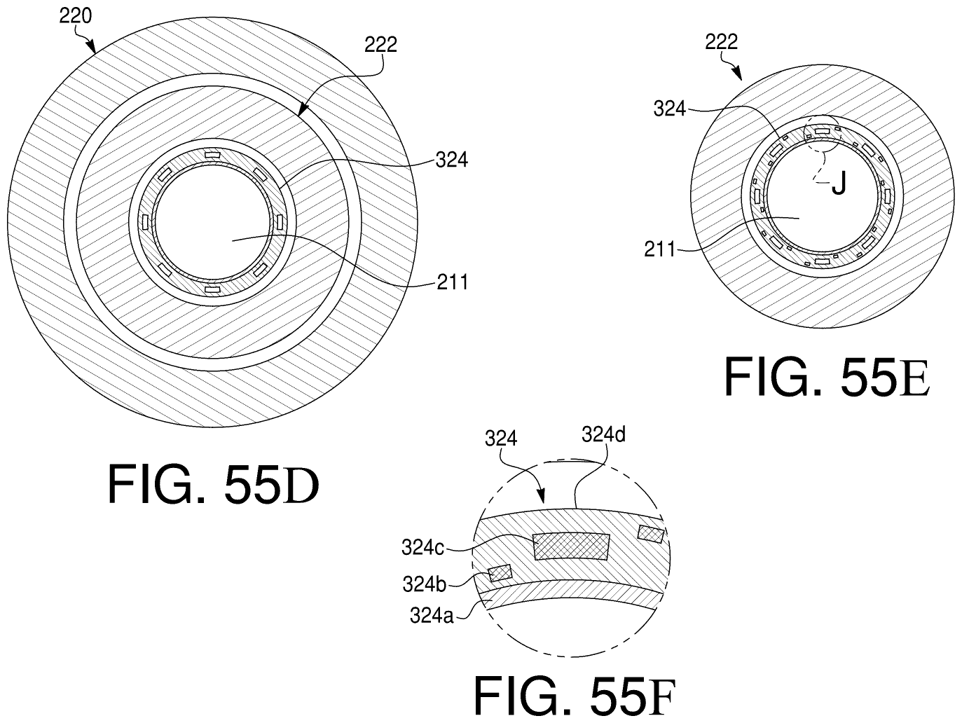

With reference to FIGS. 55A-55F, an alternative dilator 310 is shown, which may be similar in many aspects to the dilator 210 described previously. FIG. 55B is a detailed, partially sectioned view taken at circle B in FIG. 55A, FIG. 55C is a detailed, partially sectioned view taken at circle C in FIG. 55A, FIG. 55D is a cross-sectional view taken along line D-D in FIG. 55A, FIG. 55E is a cross-sectional view taken along line E-E in FIG. 55A, and FIG. 55F is a detailed view taken at circle F in FIG. 55E. In general, common elements between dilators 210 and 310 are numbered the same. As compared to the prior embodiment, dilator 310 may have a different composite configuration.

With reference to FIG. 55D, the proximal portion 216A of the dilator shaft 216 may include an outer jacket 220, an inner jacket 222 and an alternative tubular stiffening member 324. With reference to FIG. 55E, the distal portion 216B of the dilator shaft 216 may exclude the outer jacket 220 and thus include the inner jacket 222 and the alternative tubular stiffening member 324. As seen in FIG. 55F, the tubular stiffening member 324 may include an inner polymer layer 324a, a braided metallic layer 324b, one or more longitudinal metallic members 324c, and an outer polymeric layer 324d, wherein the polymeric layers encapsulate the metallic layers. The inner and outer polymeric layers 324a and 324d may include polyimide, for example. The braided metallic layer 324b may include two layers of 16 filar stainless steel ribbon braided in opposite directions, for example. The metallic longitudinal members 324c may include triaxial stainless steel ribbon, for example.

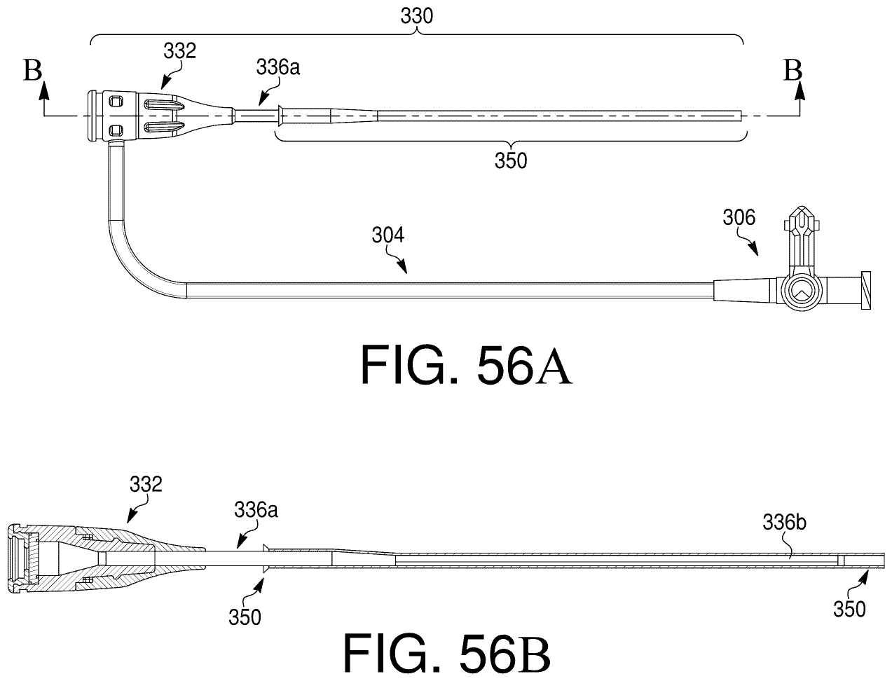

With reference to FIG. 56A, which shows the sheath 330, and FIG. 56B, which shows a longitudinal sectional view taken along line B-B in FIG. 56A, a sheath protector 350 may be utilized to aid in retaining the collapsed profile of the sheath 330 when packaged and in transit and subsequently removed before use. The sheath protector 350 may include a tubular structure formed of a low friction material such as PE. The sheath protector 350 may have a length sufficient to cover the non-expandable 336A and expandable 336B portions of the sheath shaft. The sheath protector 350 may a profile corresponding to the sheath shaft 336 wherein the inside diameter of the sheath protector 350 corresponds to the outside diameter of the sheath shaft 336 as it varies along its length. The sheath protector 350 may also include a proximal flare to facilitate loading.

With reference to FIGS. 57A and 58, flow charts of example manufacturing methods for the sheaths and dilators described herein, are shown respectively. The sheaths and dilators described herein may be, at least in part, made by conventional means known in the art. The following includes manufacturing techniques not known in the art.

With reference to FIG. 57A, the sheath may be made by a process of jacketing, folding, creasing, shape setting, tipping, hub over-molding, hydrophilic coating and assembly. FIGS. 57B-57H illustrate various tools and cross-sectional views during different stages of the manufacturing process. The jacketing step may be performed by laminating or over-extruding, for example.

In the laminating jacketing step, a liner 240 is placed and stretched over a build mandrel 370 having a constant outside diameter, as seen in FIG. 57B. Alternatively, the mandrel 370 may be supplied with a film cast liner 240 mounted on it. Extrusions are cut to form the outer jacket 242 on the proximal (non-expandable) and distal (expandable) segments. The jacket 242 covers the proximal segment fully-circumferentially and the distal segment partially-circumferentially leaving a window 242a without jacket material that will become the collapsible and expandable portion of the sheath where the liner 240 is folded. A hinge forming mandrel 372 is placed diametrically across from the window 242a. The forming mandrel 372 will form the hinge 246 of the sheath that inhibits/slows down the recovery of the outer jacket 242. A heat shrink tube 374 is placed over the assembly. Heat is applied to assembly leading to the recovery of the heat shrink to hold the assembly pieces in place. Heat is again applied to the assembly laminating the outer jacket 242 to the liner 240. At the same time, the forming mandrel 372 is pushed into the outer jacket 242, forming the hinge 246 feature. The heat shrink and mandrel are removed from the assembly after cooling.

In the alternative over-extrusion jacketing step, an etched liner is supplied over a mandrel in a continuous length. The outer jacket is directly extruded over the liner in a continuous manner. The outer jacket extrusion can be profiled where the hinge feature is formed in conjunction during the over-extrusion process. The outer jacket can be offset as well, resulting in an eccentric extrusion profile where the outer jacket is thicker on one side, thinner on the other. In another instance, the etched liner could be supplied with a unetched segment that matches the profile of the web (expandable liner) segment of the sheath. The unetched (or masked) segment prevents polymer adhesion during the over-extrusion process and results in a segment of no polymer. A segment of the outer jacket is removed by using an excimer laser to form the window. The extruded assembly is masked while exposed to the laser to control the size and shape of the ablated segment. The outer jacket is ablated while leaving the underlying liner intact. This operation can be fully automated and done in continuous lengths. In the case where the hinge is not formed in the over-extrusions process or needs to be re-formed or re-shaped, a secondary process using a forming tool can be applied. The forming tool can be hard tooling (SS, aluminum, titanium) that can have a PTFE coated internal surfaces or soft tooling (polymer or silicone) or a combination of both hard and soft tooling). The re-form or re-shape is accomplished by sleeving the tooling over sheath with the inside diameter support by a mandrel. Heat-shrink tubing is placed over the forming tooling and heat is applied to melt or reflow the sheath. The heat-shrink, forming tooling, and mandrel are removed once the assembly is cooled.

In the fold and crease step, a folding mandrel 376 is inserted into the jacketed sheath sub assembly (240, 242, 242a) made in the prior step. As seen in FIG. 57C, the folding mandrel 376 includes a distal groove or trough portion corresponding to the expandable portion of the sheath, and a proximal solid cross-section portion corresponding to the non-expandable portion of the sheath. At the window 242a, the liner 240 is folded inward towards the groove as shown in FIGS. 57D and 57E. This can be done by running the liner along the length of the groove with a fine point tool 378. The folding mandrel is then removed from the sheath when cooled.

In a creasing and shape set step, a heat set mandrel 386 is inserted into the sheath. As seen in FIG. 57F, the heat set mandrel has a solid cross-section and profile that matches the sheath inside diameter in a delivery configuration (with a 6 F proximal and 4 F distal profile, for example). The heat set mandrel 386 offsets the liner to one side of the lumen as seen in FIG. 57G. Heat shrink tube 374 may be laid or slid over the assembly and heat may be applied to shape set the sheath to its distal profile as shown in FIG. 57H. The recovery force from the heat shrink creases the folded liner in this step, leading to permanent deformation and crazing. This creates the fold direction bias in the expandable section of the sheath. The heat shrink and mandrel are removed once the assembly cools.

In a tipping step, a tipping mandrel is inserted to support the distal inside diameter of the sheath. Heat is applied approximately 3 mm from the distal tip to form and seal the distal tip. The seal is formed by reflowing and joining material on both sides of the folded liner in the window. The sealed tip creates a smooth transition between the tip and the sheath body as well as between the sheath and the dilator. The sealed tip also creates a robust tip for the expandable portion while puncturing through the arteriotomy. Tipping may be done by means of reflow where a heat shrink tube is applied over the heat, by means of RF where the tip is inserted into a tip die and axial pressure is applied to the sheath while RF energy is applied to the forming die, by means of glass die where heat is applied to the glass die and axial pressure is applied to the sheath to form the tip. Mold release may be applied to aid in the forming of the tip. The tipping mandrel is removed from the sheath once the tip is formed and the assembly cools.

In a hub over-molding step, a core pin is inserted into the sheath to support the proximal inside diameter. The sheath assembly is placed in the mold cavity of the injection molder. The injection molder is cycled to over-mold the hub onto sheath. The core pin may then be removed from sheath.

In a hydrophilic coating step, a mandrel or plug may be inserted into the sheath to mask the inside diameter of the sheath. A portion, e.g., proximal half, of the non-expandable portion of the sheath may also be masked. A hydrophilic coating may be applied to the sheath. This can be done by means of wipe, spray dispersion, or dip coating. The mandrel or plug is removed once the hydrophilic coating is fully cured.

In an assembly step, the flush port tubing and stopcock are connected to the flush port on the hub. This can be done by means of friction fit using a barb, by means of adhesive, or by means of solvent bonding between the tubing and the flush port on the hub. The hemostasis valve is assembled into the proximal end of the hub, and the snap cap is inserted onto hub. The compression fit between the snap cap, hemostasis valve, and hub creates a hemostasis seal within the hub cavity. A sheath protector is then placed over the sheath. This protector maintains the low profile of the sheath throughout the sterilization process and aging of the product.

With reference to FIG. 58, the dilator may be made by a process of tipping extrusion, bond extrusions, hub over-molding and assembly.

In the tipping extrusion step, a tipping mandrel may be inserted to support distal inside diameter of the inner dilator extrusion 222. Heat is applied to the distal 0.75 in. of the tip to form the distal tip 218. The formed distal tip 218 creates a seamless transition to the guidewire while puncturing through the arteriotomy. Tipping can be done by means of reflow where a heat shrink is applied over the heat, by means of RF where the tip is inserted into a tip die and axial pressure is applied to the dilator while RF energy is applied to the forming die, by means of glass die where heat is applied to the glass die and axial pressure is applied to the dilator to form the tip. Mold release may be applied to aid in the forming of the tip. In the hub over-molding step, the mandrel is removed from the dilator once the tip is formed and the assembly cools.

In the bond extrusions step, a stiffening member 324 is inserted into the tipped 218 dilator extrusion 222. The stiffening member 324 can be made of a braided composite tubing, a rigid polymer extrusion, a rigid hypotube, or a laser-cut hypotube. The outer jacket 220 is slid or laid over the inner 222 extrusion. A mandrel is inserted to support the inside diameter of the stiffening member 324. Heat shrink is laid or slid over the transition that is the tapered section 216C between the outer jacket 220 and inner jacket 222. Heat is applied over the heat shrink. Heat can be applied by means of heat gun (forced convection), by means of split die (conduction), by means of RF, or by means of ultrasound welding. This forms a smooth transition along the tapered section 216C between the proximal 216A and distal 216B sections. At the same time, the heat bonds the outer and inner extrusions and stiffening member at the taper junction. The heat shrink and mandrel are removed once the assembly cools.

In the hub over-molding step, a core pin is inserted into a proximal inside diameter of the stiffening member 324 to support it and keep it patent. The assembly is placed in the mold cavity of the injection molder. The injection molder is cycled to over mold the hub onto the dilator. The core pin is then removed from dilator. In the assembly step, snap cap is pushed onto the dilator assembly.

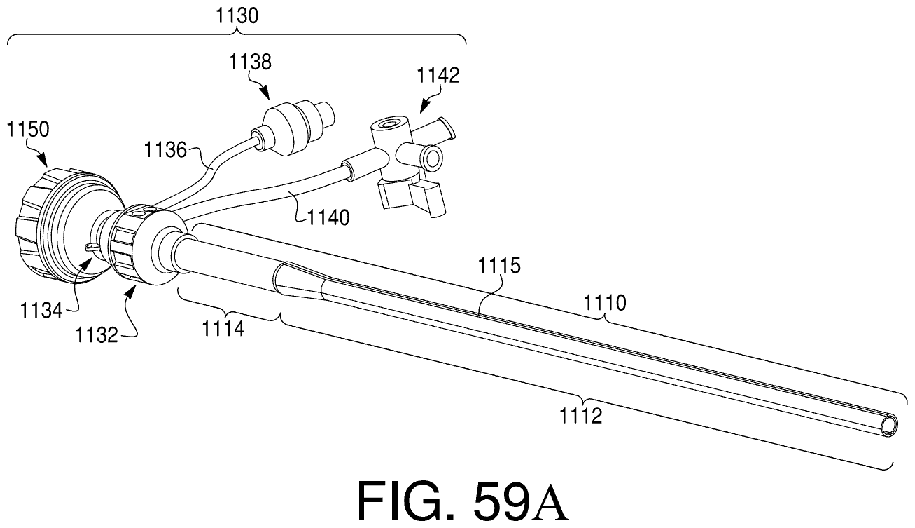

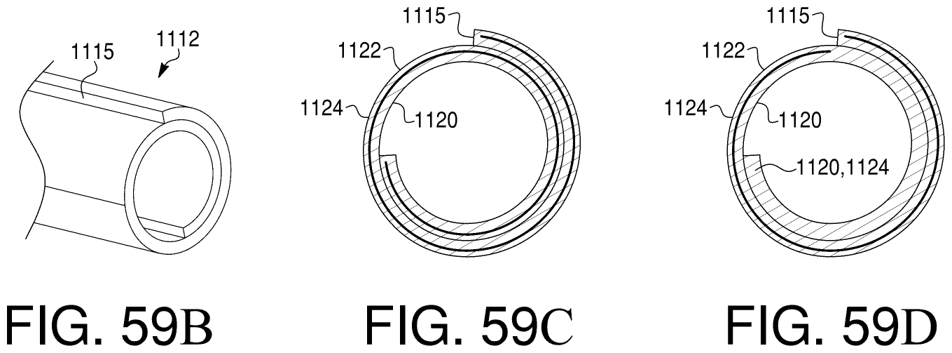

With reference to FIG. 59A, a perspective view of an expandable introducer sheath according to an embodiment of the present disclosure is shown. FIG. 59A shows the sheath body 1110 in a collapsed configuration. In this embodiment, the expandable concepts described hereinbefore may be applied to this large bore (e.g., approximately >10 F) example. In general, the expandable sheath system may include an expandable tubular sheath body 1110 having a slit 1115, a dilator (not shown) with a tapered end and optional bulbous tip, and a main hub assembly 1130 connected to a proximal end of the sheath body 1110. FIG. 59A shows the sheath body 1110 in a collapsed configuration. The sheath body 1110 may include an expandable (slit) distal segment 1112, a non-expandable (un-slit) proximal segment 1114 that has a constant diameter, and a tapered segment therebetween. Optionally, the non-expendable portion 1114 may include a proximal continuation of the expandable portion 1112 with an elastomeric tubular cover. The non-expandable portion 1114 (or the elastic tubular cover) may provide a uniform outer surface to enable effective hemostasis at the arteriotomy.

Except for the features related to expandability, other aspects may be the same or similar to the corresponding features described in U.S. patent application Ser. No. 18/644,816, filed Apr. 24, 2024, the entire disclosure of which is incorporated herein by reference.

In general, to mitigate kinking of the sheath body 1110 at this relatively larger diameter in the presence of a longitudinal slit 1115, a support structure may be desirable. Although wire coils and braids are known support structures in catheter construction, creating a slit 1115 through a wire coil or braid may create exposed wire ends that are relatively sharp. Further, creating a slit 1115 through a wire coil or braid may result in the ribs becoming disassociated from the laminate polymer layers, potentially creating embolic material. To mitigate this, a polymer may be disposed over then ends (e.g., by reflowing a polymer jacket over the end) to create an atraumatic edge. Alternatively, a serpentine ribcage support structure of metal or hard polymer may be used, with or without a spine, wherein the slit 1115 extends between the rounded ends of the serpentine ribs. The serpentine ribcage thus avoids exposed wire ends, and further interconnects the ribs such that they do not become dislodged from the laminate polymer layers. As a further alternative, a ribcage pattern may be formed in a metallic tube to have the same or similar effect. Specific examples of such are described hereinafter.

With reference to FIGS. 59B-59D, the sheath body 1110 may include, for example, a slitted composite tube including a PTFE inner layer or liner 1120, a metallic (e.g., stainless-steel or nickel titanium) ribcage 1122 disposed about the inner layer 1120, and an outer layer or jacket 1124 (e.g., polyether-block-amide) disposed over the ribcage 1122. The ribcage 1122 may extend the full circumference between the liner 1120 and jacket 1124 as shown in FIG. 59B, or the ribcage 1122 may extend only a portion of the full circumference between the liner 1120 and jacket 1124 as shown in FIG. 59D.

The sheath body 1110 may be connected to the main hub assembly 1130 via screw-type connector 1132, for example. The slit 1115 may be configured to be straight and run parallel to the longitudinal axis as shown or may be spiraled around the sheath body 1110 (e.g., one revolution every 10 cm). To facilitate visualization via fluoroscopy, the polymer of the tip may be loaded with radiopaque material (e.g., 70% Tungsten loading in 40D Pebax), or a metallic radiopaque marker band (e.g., Tungsten ring) may be incorporated into tip between the layers thereof, for example. Further details of an example slit sheath body 1110 configuration are described hereinafter. The slit sheath 1110 may be available in sizes (diameters) ranging from 10 F to 26 F and lengths of 15 cm, 33 cm, 45 cm or 65 cm, for example.

The dilator (not shown) may include a relatively rigid LDPE tube, for example, with a tapered bulbous distal end, a constant diameter main body portion, and a proximal hub. The lumen of the dilator extends the entire length thereof to accommodate a guide wire (e.g., 0.035″ guidewire compatible, not shown), over which the assembly is delivered. The main body portion of the dilator may have an outer diameter that closely matches the inside diameter of the sheath body 1110, with a small gap to allow relative movement therebetween. The tapered bulbous distal end of the dilator provides gradual dilatation of the vascular puncture site and a smooth transition from the guidewire to the sheath distal tip. The dilator hub may include an interlocking feature that mates with, and releasably secures to, the main hub assembly 1130. To facilitate visualization via fluoroscopy, the dilator may be loaded with a radiopaque material such as barium sulfate.

The main hub assembly 1130 generally includes a housing 1134, formed of an injection molded polymer, for example. The sheath-to-hub connector 1132 may be secured to a distal aspect of the housing 1134. A proximal aspect of the housing 1134 may contain a hemostatic valve (not visible). Generally, the hemostatic valve provides a seal around inserted devices to minimize blood loss. The hemostatic valve may include a relatively rigid or non-collapsible valve body and a collapsible sleeve (e.g., ePTFE, FEP, or a laminate thereof) mounted at both ends to the valve body. The sleeve may be compressed by pressurizing the space between the valve body and the sleeve. Pressurizing the sleeve may accomplished by connecting a saline-filled syringe to, and injecting saline through, a valve line 1136 via a connector 1138 (e.g., needleless valve, stopcock, etc.). The connector 1138 associated with the valve line 1136 may be configured differently than the connector 1142 associated with the flush line 1140 so as to avoid confusion between the connectors and mitigate human error.

The hub assembly 1130 may also include a flush line 1140 with a connector (e.g., 3-way stop cock) for connection to a syringe or power injector to facilitate the injection or removal of gases such as air or liquids such as saline, contrast media, etc. to/from the introducer sheath and/or the vasculature. The hub housing 1134 may define a transparent bubble chamber 1144 to visualize bubbles that may be inadvertently introduced when inserting large devices into the hub assembly 1130, whereby such bubbles may be removed via the flush line 1140. To facilitate this, the flush line may be connected to a vertical apex of the bubble chamber 1144.

The hub may further include a pressure regulator 1150 operably connected to the valve assembly. The pressure regulator 1150 may reduce the magnitude of increased pressure in the space between the valve body and the collapsible sleeve as devices are inserted into the valve assembly. By reducing the magnitude of increased pressure, the pressure regulator 1150 can mitigate the degree of increased friction imposed by the sleeve on devices inserted into the valve assembly. By mitigating friction, inserted devices may be freer to manipulate or otherwise move relative to the sheath assembly, thus allowing better control thereof.

The pressure regulator 1150 may include a housing defining or containing a sealed chamber. The outside of the sealed chamber may be in fluid communication with the valve, and the inside of the sealed chamber may be fluidly isolated from the valve. The inside of the chamber may contain a compressible component (e.g., a gas such as air) that is more compressible than the fluid (e.g., a liquid such as saline). For example, the sealed chamber may include a closed-cell foam wherein the inside of the cells contain air. Alternatively, a deflectable diaphragm supported by a rigid housing may define the sealed chamber, wherein the chamber is filled air. As a further alternative, the sealed chamber may include an air-filled bladder. Another alternative is a piston-camber arrangement wherein a plunger with a sliding seal resides within a chamber filled with air. In each case, the inside of the sealed chamber may be fluidly isolated from the valve. Fluid isolation may be beneficial when using gas in the pressure regulator to avoid introducing the gas into the vascular system in the event of a valve failure.

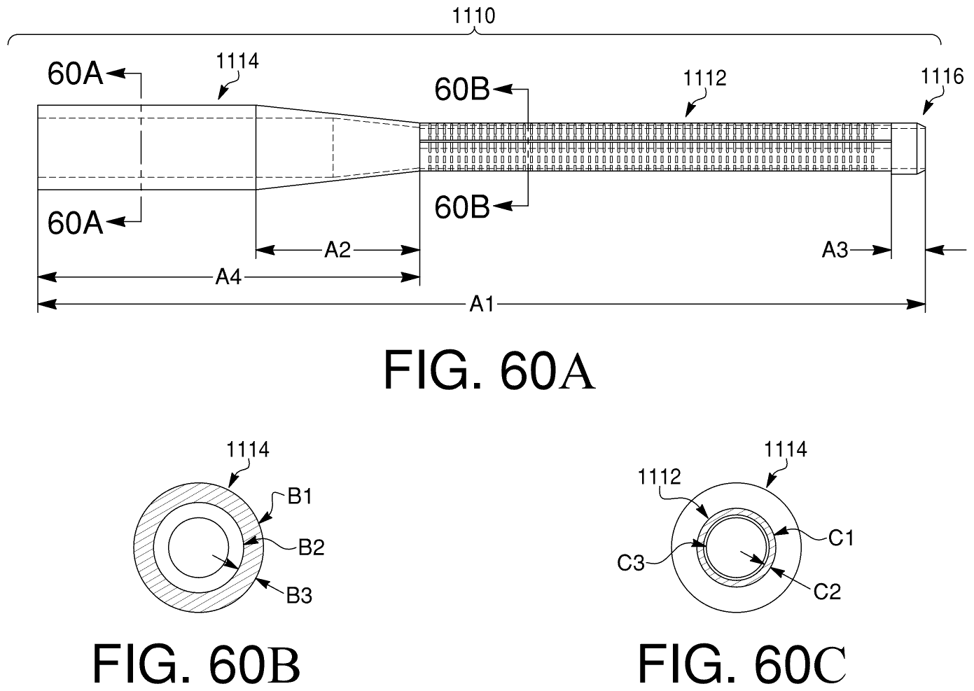

With reference to FIG. 60A, an example construction of the slit expandable sheath body 1110 is shown schematically. FIG. 60B is a cross-sectional view taken along line B-B, and FIG. 60C is a cross-sectional view taken along line C-C. Listed materials, dimensions and construction notes are given by way of example. In this example embodiment, the sheath body 1110 may include an expandable (slit) distal segment 1112, a non-expandable (un-slit) proximal segment 1114 that has a constant diameter, and a tapered segment therebetween. In this example embodiment, sheath body 1110 may have a longitudinal length A1 of approximately 37 cm. Expandable (slit) distal segment 1112 may have a longitudinal length of approximately 29 cm. Non-expandable (un-slit) proximal segment 114 may have a longitudinal length of approximately 7.125 cm. Tapered segment may have a length A2 of approximately 0.875 cm. Non-expandable (un-slit) proximal segment 1114 and tapered segment may have a combined longitudinal length A4 of approximately 8 cm. Splittable polymer tip 1116 may have a longitudinal length A3 of approximately 5 mm. The laminate construction may include a thin, low friction inner liner including film cast PTFE with a wall thickness of approximately 0.0015 inches, for example. A tie layer may be disposed on the inner liner with a thickness of approximately 0.005 inches, and a support structure may be disposed around the tie layer. The PTFE liner and the tie layer may terminate at approximately 1 mm from the splittable polymer tip 1116. In this example, the ribcage may include a serpentine wire form of approximately 0.010 inch diameter round stainless steel wire. The ribcage portion may terminate at approximately 3 mm away from the splittable polymer tip 1116. A relatively soft polymer such as 25D Pebax may be disposed over the ribcage and reflowed with a wall thickness of approximately 0.010 inches. The 25D Pebax disposed over the ribcage may terminate within the tapered segment. A longitudinal slit may then be formed (e.g., cut) through the polymer layers between the ends of the ribcage. The expandable distal segment 1112 may be rolled along its longitudinal axis into a collapsed configuration as seen in FIG. 60B. A splittable polymer tip 1116 may be secured to the distal end. Splittable polymer tip 1116 may include a 40D Pebax, Prox Jacket lining loaded with approximately 70% tungsten with a wall thickness of approximately 0.015 inches. Tapered segment may include a 72D Pebax, Prox Jacket lining with a wall thickness of 0.020 inches. The distal segment 1112 may be reflowed to create a splittable bond between the overlapping jacket portions to retain the collapsed configuration. The overlapping jacket portions may have an approximately 180 degree twist. In the collapsed configuration, the expandable sheath body 1110 may be inserted into the access artery or vein and partially expanded by retracting a dilator with a bulbous tip to split the tip 1116 and distal segment 1112. Further expansion may occur by inserting larger bore devices therethrough.

To maintain the distal portion of the sheath body 1112 in a collapsed configuration during insertion and to maintain a smooth transition between the dilator tip and the distal tip 1116 of the sheath body 1112, the distal tip 1116 of the sheath body 1112 may be configured such that the edges of the slit are temporarily bonded in the area where they overlap. Such temporary bond may include a reflow of material over a short length, for example. The temporary bond may be broken upon insertion of a larger device (e.g., bulbous dilator) to expand the expandable portion 1112 of the sheath body.

As shown in FIG. 60B, the lining of non-expandable (un-slit) proximal segment 1114 may have an inner diameter B2 of approximately 0.325 inches and an outer diameter B1 of approximately 0.365 inches. Non-expandable (un-slit) proximal segment 1114 may have a wall thickness B3 of approximately 0.020 inches. As shown in FIG. 60C, the lining of expandable (slit) distal segment 1112 may include an inner diameter C3 of approximately 0.183 inches (14 FR) and outer diameter C1 of approximately 0.240 inches. The expandable (slit) distal segment 1112 may include a wall thickness C2 from approximately 0.020 inches to 0.034 inches.

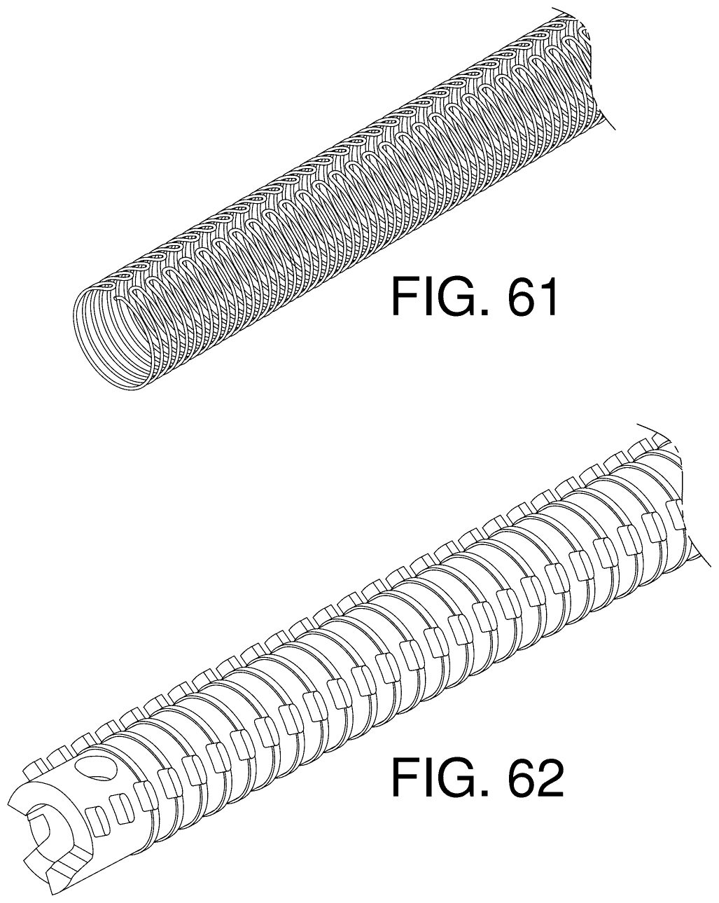

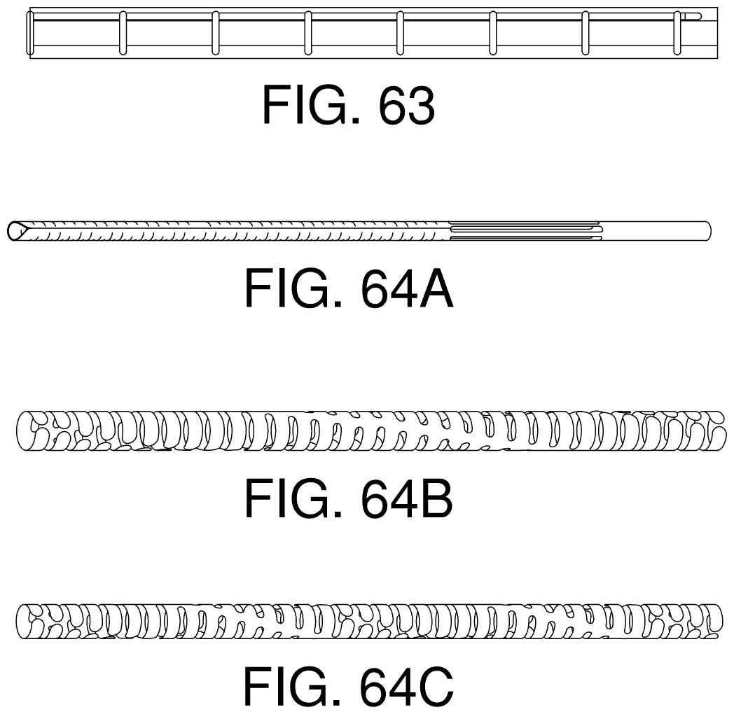

FIG. 61 is a schematic perspective view of a serpentine ribcage that may be disposed between polymer layers. Note the return loop for each rib and the gap between rib ends to accommodate a slit. FIG. 62 is a schematic perspective view of a serpentine ribcage formed on a fixture including alternating posts along a rod. FIG. 63 is an example of a ribcage with each rib connected to a spine. FIGS. 64A, 64B and 64C are examples of a ribcage patterns formed in metallic tubes wherein the ribs are integrally connected.

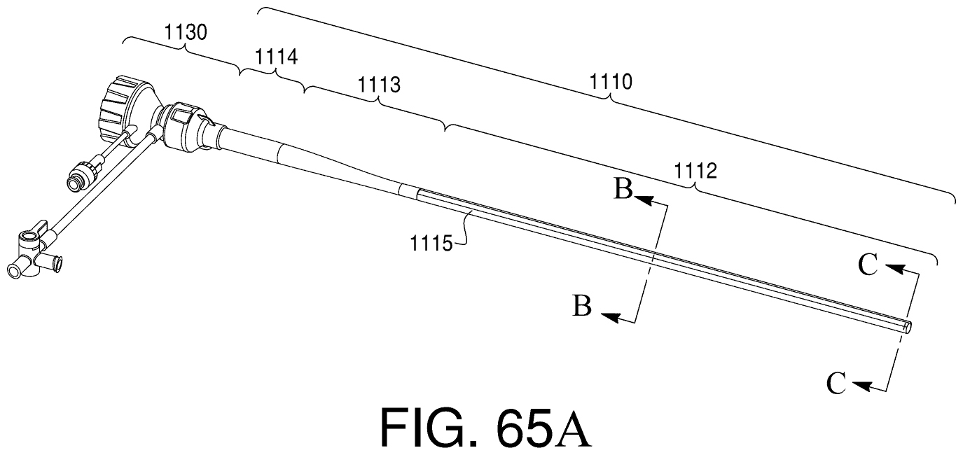

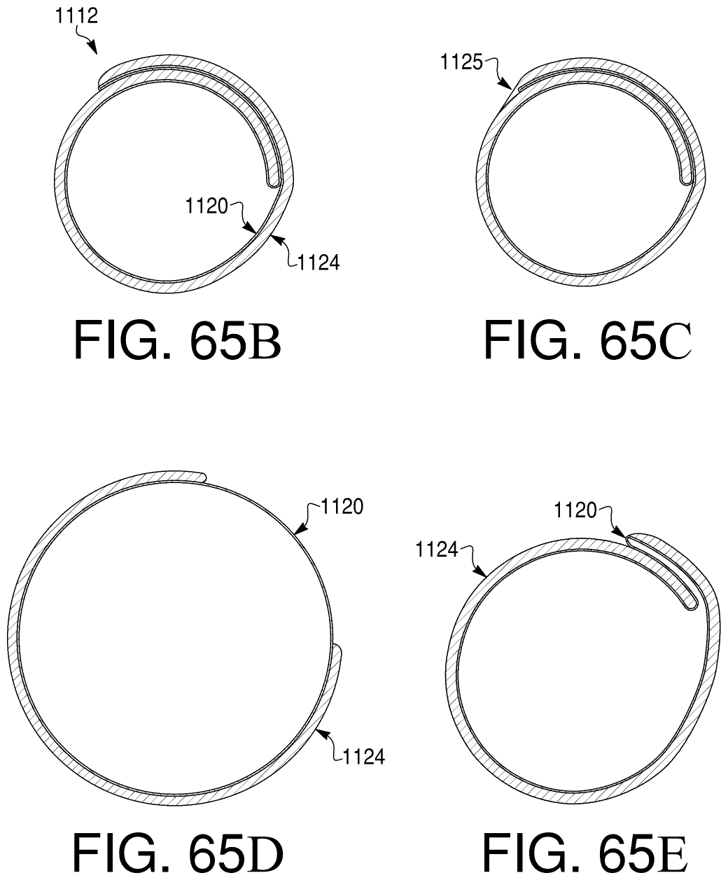

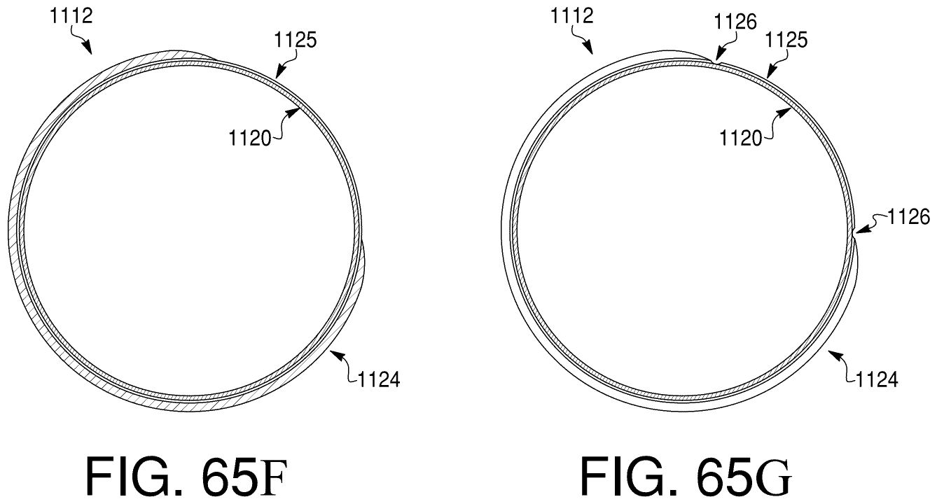

With reference to FIG. 65A, a perspective view of an alternative expandable sheath is shown in a collapsed state, which may be similar in many aspects to expandable introducer sheath described with reference to FIG. 59A. In general, common elements between the introducer sheaths described with reference to FIGS. 59A and 65A are numbered the same. FIG. 65B is a cross-sectional view of the expandable shaft section 1112 taken along line B-B in FIG. 65A, and FIG. 65C is a cross-sectional view of the tip of the expandable shaft section 1112 taken along line C-C in FIG. 65A. FIGS. 65B and 65C shown the expandable shaft 1112 in a collapsed state, whereas FIGS. 65D and 65E show the expandable shaft 1112 in an expanded and partially recovered state, respectively.

As compared to the prior embodiment illustrated in FIG. 59A, the introducer sheath illustrated in FIG. 65A may have a slit that continues proximally through a tapered section 1113 having an elastomeric tubular cover, which may be positioned in the arteriotomy when in use. As seen in FIGS. 65B-65E, the expandable shaft 1112 may include a liner 1120 and an outer jacket 1124. At the distal tip of the shaft 1112, the jacket 1124 may be reflowed to create a temporary bond 1125 to maintain the shaft in a collapsed state during delivery. After delivery, the temporary bond 1125 may be broken by delivering an enlarged payload (e.g. valve delivery device or dilator with a bulbous tip, for example) allowing the shaft 1112 to expand.

In this embodiment, the liner 1120 may include film-cast PTFE with a wall thickness approximately less than 0.002″ with an inside diameter greater than approximately 0.100″, which aids in recovery as shown in FIG. 65E. In general, ram extruded PTFE is limited to a wall thickness greater than approximately 0.002″ at an inside diameter of approximately 0.100″ or more. The film-cast PTFE liner 1120 also has a lower durometer than ram extruded PTFE rendering it softer and therefore less traumatic to the vascular wall when in use. In other words, the film-cast PTFE liner 1120 is softer and thinner than conventional ram extruded PTFE liners known in the art. The softness of the liner material allows the jacket to not meet resistance as it recovers, and the thinness of the liner promotes more recovery to its original fold geometry, rather than creating a protrusion, as shown in FIG. 65E (note that the liner 1120 recovers toward the inside lumen rather than protruding outward between the edges of the jacket 1124).

With reference to FIGS. 65F and 65G, alternative constructions of the sheath shaft 1112 are shown in cross-section in the expanded state. In these embodiments a thin (e.g., approximately 0.0002″ to 0.001″ wall thickness) layer or coating 1125 may be applied between the liner 1120 and the jacket 1124. Optionally, the thin layer 1125 may be a thin-walled integral continuation of the jacket 1125 such that the liner 1120 never extends beyond the jacket 1124 during expansion or recovery. With reference to FIG. 65G, the thin layer may have material removed to form longitudinal hinges 1126. The hinges 1126 may allow the liner 1120 and thin layer 1125 to act like a rigid swinging gate wherein the edges of the slit jacket 1124 remain at a fixed distance during expansion and recovery.

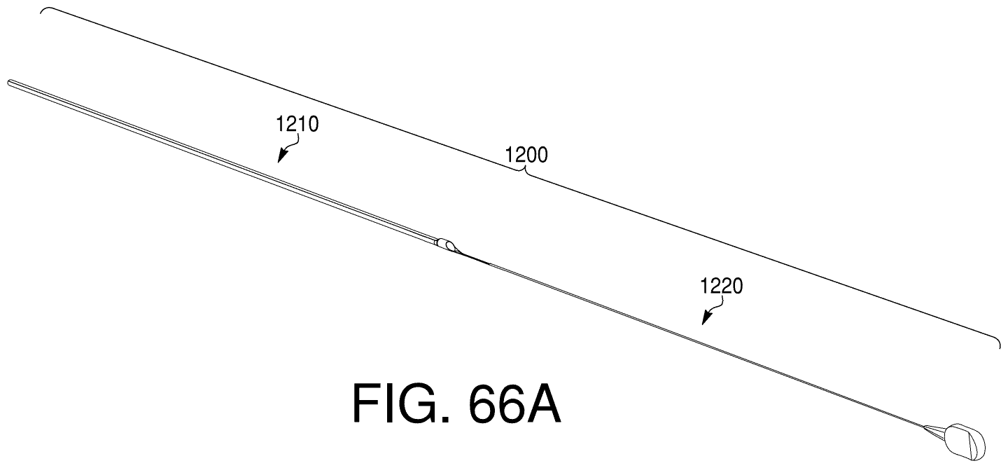

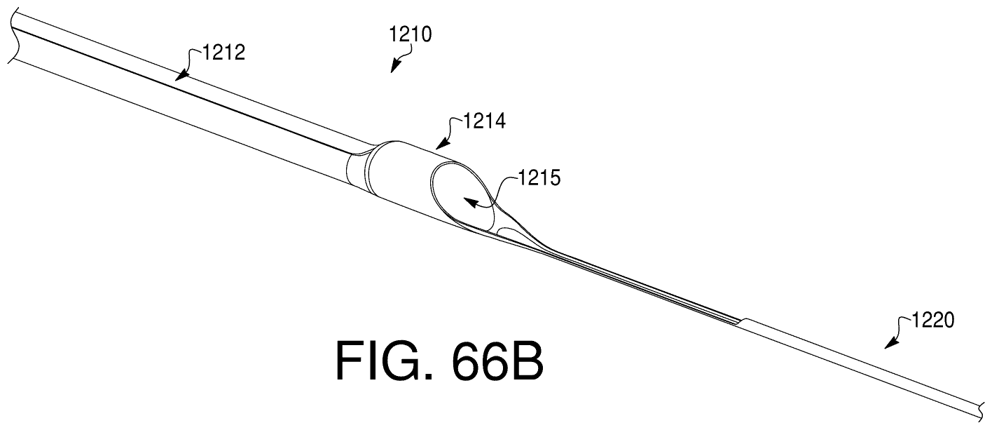

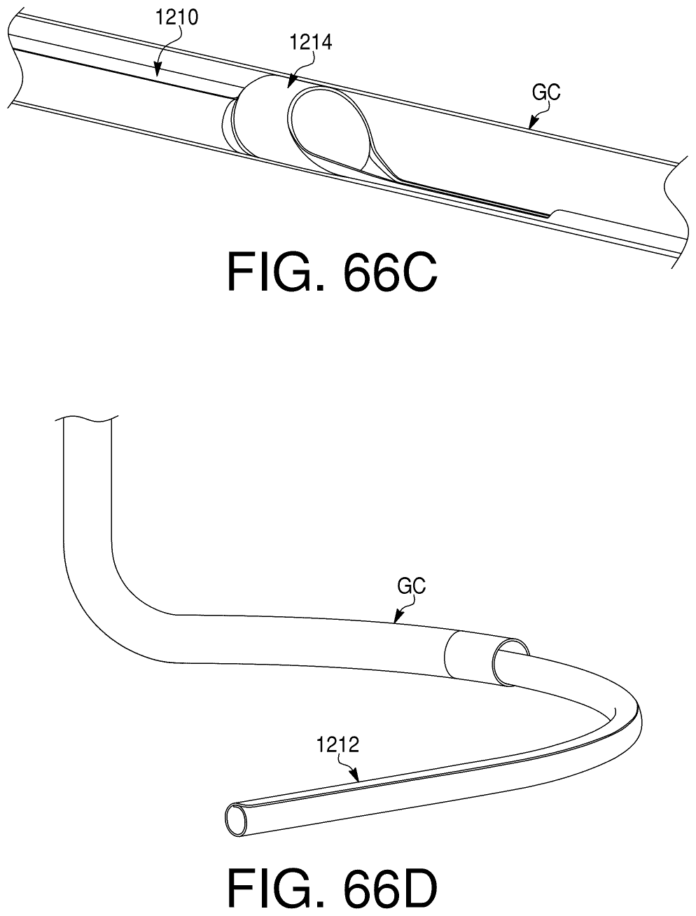

With reference to FIGS. 66A to 66D, perspective views of an expandable guide catheter extension (GCE) (also known as guide extension catheter, or GEC) 1200 are shown according to an embodiment of the present disclosure. In this embodiment, the expandable concepts described hereinbefore are applied to this GCE example. With specific reference to FIG. 66A, the GCE 1200 may include a slit expandable tubular segment or body 1210 and a push member 1220 connected to a proximal end of the tubular body 1210. With specific reference to FIG. 66B, the tubular body 1210 may include an expandable distal segment or portion 1212 and a proximal un-slit, constant diameter segment or portion 1214. As seen in FIGS. 66C and 66D, when the expandable GCE 1200 is disposed in a guide catheter (GC), the proximal segment 1214 remains inside the GC and the distal segment 1212 extends out the distal end of the GC.

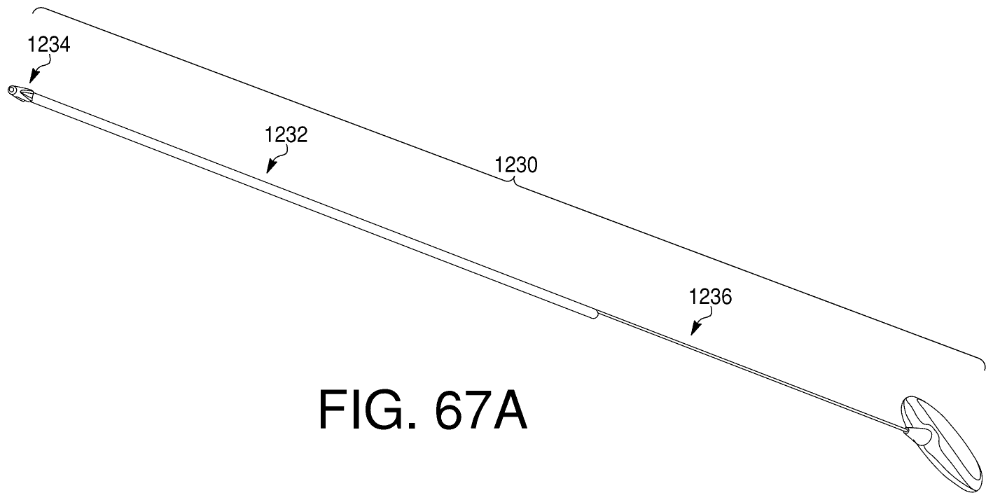

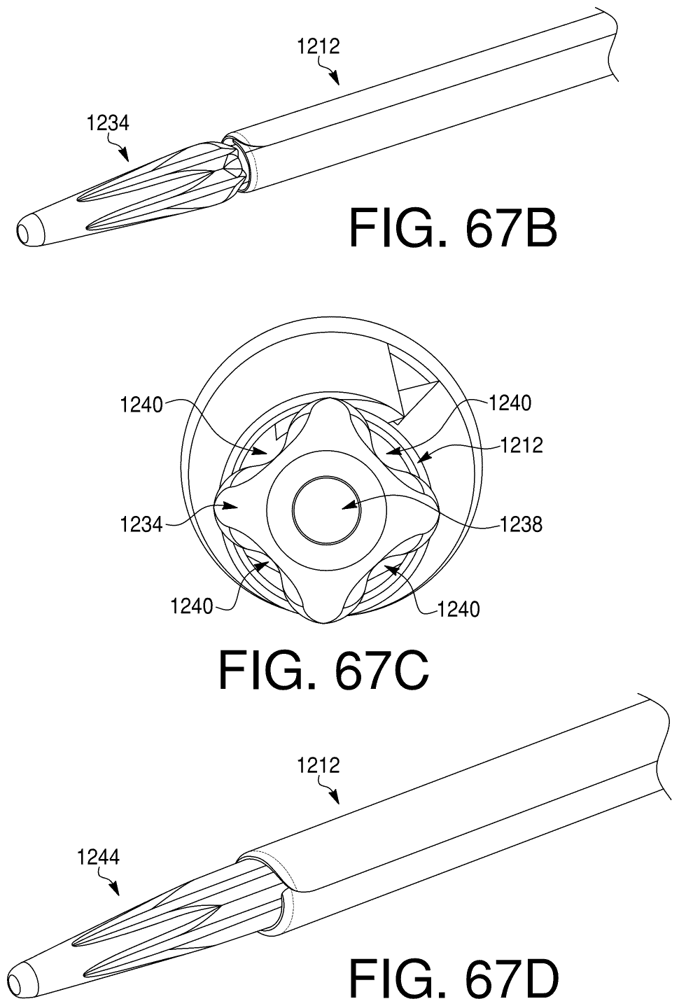

With reference to FIGS. 67A to 67D, the GCE 1200 may be disposed over a dilator 1230 for advancement through the GC and vasculature. With specific reference to FIG. 67A, the dilator 1230 may include a distal segment 1232 with a bulbous tip 1234, and a push member 1236. The dilator distal segment 1232 may be disposed in the tubular body 1210 of the GCE 1200 such that the bulbous tip 1234 extends out of the distal end of the expandable portion 1212 as shown in FIG. 67B. As shown in FIG. 67C, the distal segment 1232 and bulbous tip 1234 may define a guidewire lumen 1238 extending therethrough such that the dilator 1230 and GCE 1200 may be advanced over a guidewire as a system. The bulbous tip 1234 may have a cross-sectional shape that is other-than-round (e.g., triangular, square, clover leaf, etc.) to define vent channels 1240. Without vent channels 1240, movement of the dilator distal segment 1232 in the GCE tubular segment 1210 may tend to act as a piston in a chamber, wherein proximal withdrawal of the dilator distal segment 1232 could vacuum in blood into the GCE tubular segment 1210, potentially collapsing the vasculature in which it resides. In the presence of such a vacuum, the vent channels 1240 allow blood to flow distally into the vasculature to prevent collapse thereof. Optionally, rather than a bulbous tip 1234, a non-bulbous tip 1244 may be used, wherein the tip 1244 may have the same diameter as the rest of the dilator segment 1232 as shown in FIG. 67D.

The distal end of the expandable distal segment 1212 of the GCE 1200 may be positioned just proximal of the target treatment site. Alternatively, given the smaller collapsed diameter, the collapsed distal segment 1212 may be placed across the treatment site. When an interventional device such as a balloon angioplasty catheter or a stent delivery catheter is advanced in the GC, the distal end of the interventional device passes through the opening 1215 defined by the proximal segment 1214, through the distal segment 1212 causing it to expand, and to the treatment site. For treatment by balloon angioplasty or stent delivery, the GCE 1200 may remain in place if positioned proximal of the treatment site, or it may be retracted proximally if positioned across the treatment site.

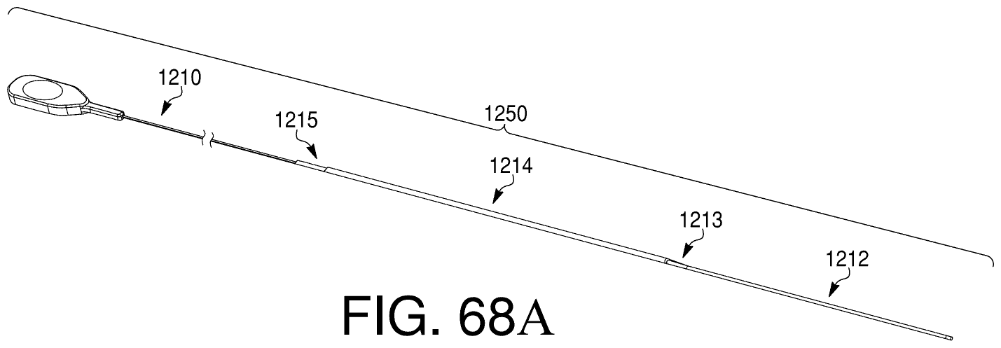

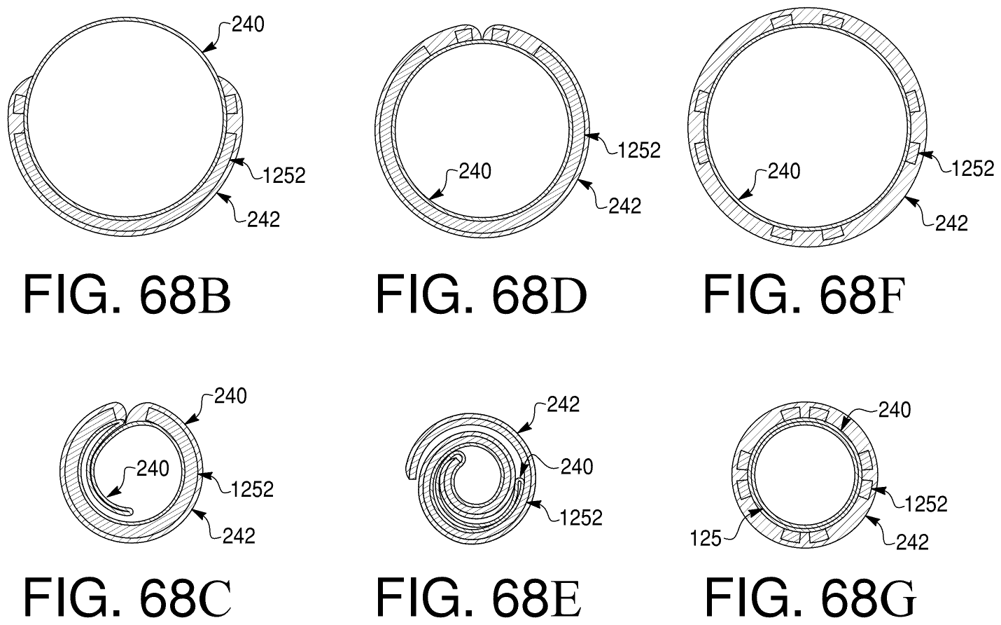

With reference to FIG. 68A, an alternative expandable GCE 1250 is shown in perspective view, which may be similar in many aspects to the expandable GCE 1200 described previously. In general, common elements between GCEs 1200 and 1250 are numbered the same. As compared to the prior embodiment, GCE 1250 may have a longer non-expandable portion 1214, a longer transition or tapered section 1213 between the non-expandable section 1214 and the expandable segment 1212 and a proximal opening 1215 defined by a half-pipe, for example.

Because the expandable portion 1212 may be deployed in tortuous vasculature, a reinforcement member may be incorporated into the expandable portion 1212 to mitigate ovalling and kinking or to otherwise maintain a round lumen. FIGS. 68B-68G illustrate cross-sectional views of the expandable portion 1212 with various reinforcement structures and various ways to expand and collapse. FIGS. 68B, 68D and 68F show the expandable portion 1212 is the expanded state, whereas FIGS. 68C, 68E and 68G show the expandable portion 1212 in a collapsed state.

With specific reference to FIGS. 68B and 68C, the expandable portion 1212 of the GCE shaft may include a fully circumferential inner liner 240, a semi-circumferential outer jacket 242 and an embedded semi-circumferential reinforcement structure 1252 such as a serpentine coil or laser-cut tube as described elsewhere herein. In this embodiment, the liner 240 may be folded as described previously and shown in FIG. 68C.

With specific reference to FIGS. 68D and 68E, the expandable portion 1212 of the GCE shaft may similarly include a fully circumferential inner liner 240, an outer jacket 242 with a slit, and an embedded semi-circumferential reinforcement structure 1252. However, in this embodiment, the liner 240 is not adhered to the jacket along the entire circumference. Rather, the liner 240 may separate from the jacket 242 across the slit, thus enabling the liner 240 to be folded toward the lumen as described before, and the jacket 242 with the reinforcement structure 1252 embedded therein to be rolled about the longitudinal axis and shown in FIG. 68D.

With specific reference to FIGS. 68F and 68G, the expandable portion 1212 of the GCE shaft may include a fully circumferential inner liner 240, a fully circumferential outer jacket 242, and an embedded, fully circumferential, self-expandable, stent-like reinforcement structure 1254. In this embodiment, the liner 240 and jacket 242 may be formed of an elastomeric material to permit expansion. The reinforcement structure 1254 maybe be formed of a super-elastic metal such as nitinol that is configured to self-expand when unconstrained. To constrain the super-elastic reinforcement structure 1254 in a collapsed state, a coil 1256 may be wrapped around the reinforcement structure 1254 as shown in FIG. 68G. Upon removal of the coil 1256, for example by pulling it proximally with a pull-wire such that it unravels from the composite structure, the self-expanding reinforcement structure 1254 expands as does the elastomeric liner 240 and elastomeric jacket 242 as shown in FIG. 68F.

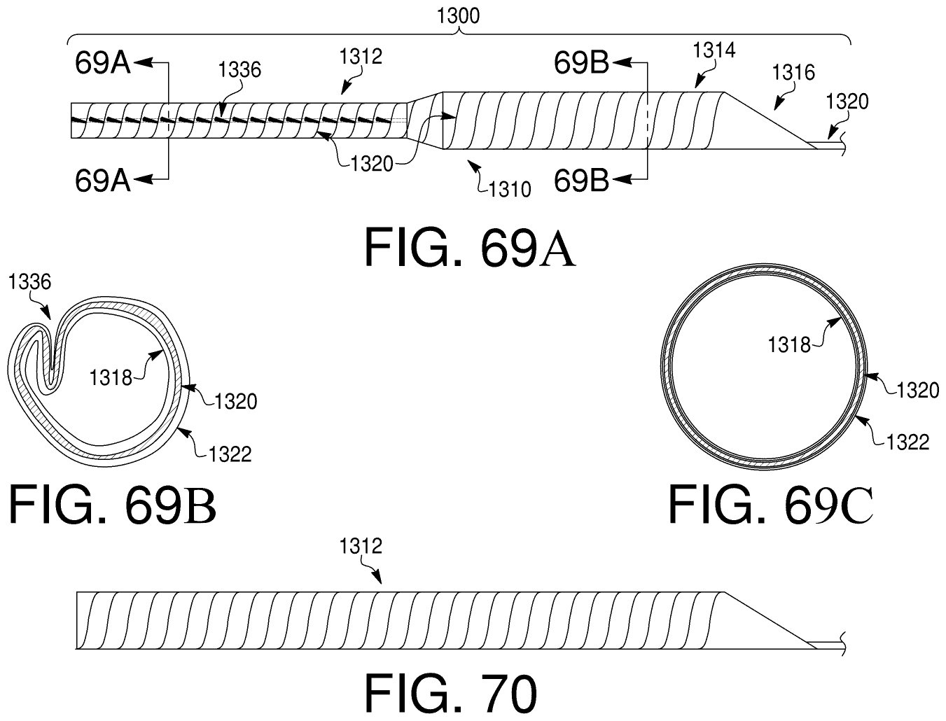

Yet another embodiment of an expandable guide extension catheter 1300 is shown in FIGS. 69A and 70. Expandable guide extension catheter 1300 may include a pushable shaft portion 1320, a distal tubular portion 1310, and a side opening portion 1316. The distal tubular portion 1310 may include an expandable portion 1312 and non-expandable portion 1314. Distal tubular portion 1310 may include an inner layer 1318, and outer layer 1322, and a reinforcement layer 1320, as seen in FIGS. 69B and 69C. Reinforcement layer 1320 serves to prevent kinking of the distal tubular portion 1310 during use. Reinforcement layer 1320 may be formed from a coil or similar structure, of a malleable material, such as stainless steel.

Expandable portion 1312 may be formed by forming an in-fold 1336 to the multi-layered expandable portion 1312, reducing the diameter. The in-fold 1336 may be expanded by an expansion device (not shown) such as a dilator or balloon catheter. After expansion, the expandable tubular portion 1312 becomes expanded, as shown in FIG. 70.

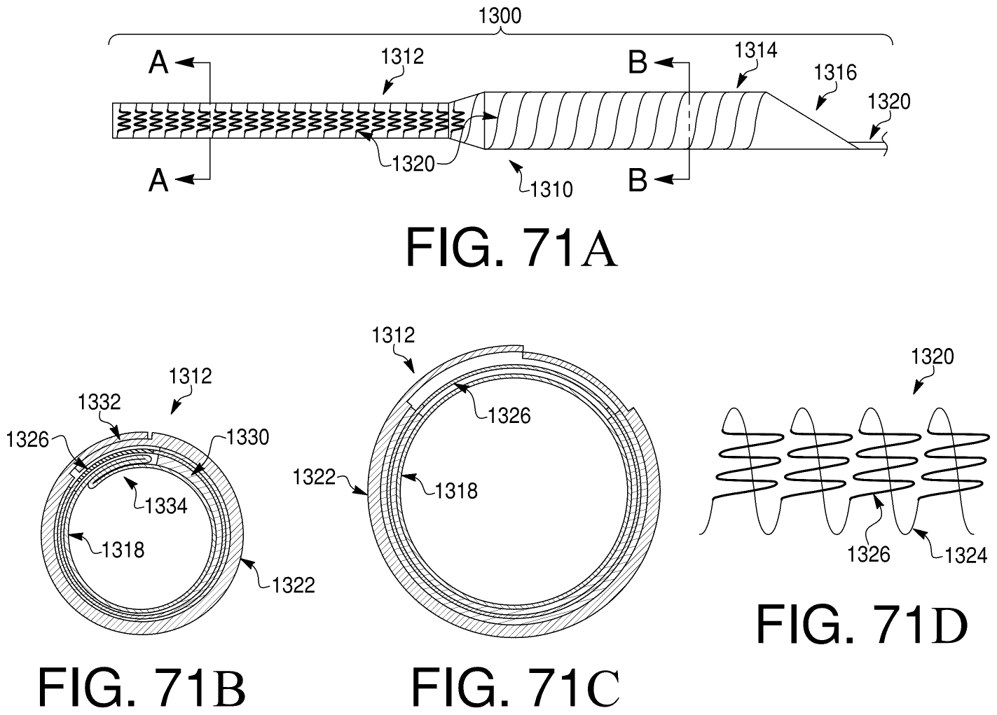

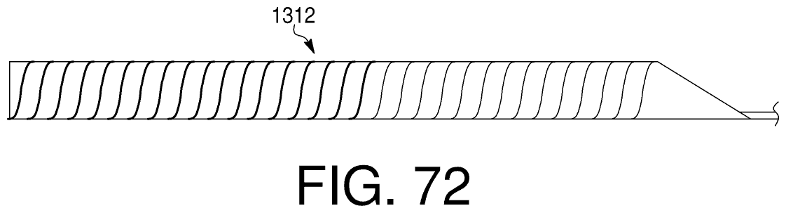

One embodiment of an expandable guide extension catheter 1300 is shown in FIGS. 71A and 72. Expandable guide extension catheter 1300 may include a pushable shaft portion 1320, a distal tubular portion 1310, and a side opening portion 1316. The distal tubular portion 1310 may include an expandable portion 1312 and non-expandable portion 1314. Distal tubular portion 1310 may include an inner layer 1318, and outer layer 1322, and a reinforcement layer 1320, as seen in FIGS. 71B and 71C. Reinforcement layer 1320 serves to prevent kinking of the distal tubular portion 1310 during use.

In the expandable portion 1312, the inner layer 1318 may be in-folded, forming an overlap 1334, as seen in FIG. 71B. Reinforcement layer 1320 may include a coil like portion 1324, and a sinusoid portion 1326 as seen in FIG. 71D. Reinforcement layer 1320 may be formed from a wire or wire-like structure of a malleable material such as stainless steel. The sinusoid portion 1326 serves to provide circumferential expandability of the reinforcement layer 1320 and can be formed of any structure which is able to plastically deform.

Referring back to FIG. 71B, the coil like portion 1324 may be embedded 1330, or otherwise secured to the inner layer 1318 and outer layer 1322. Outer layer 1322 may have an overlap region 1332, which may facilitate circumferential expansion. Sinusoidal portion 1326 may not be embedded or otherwise secured to the inner layer 1318 and outer layer 1322. The lack of embedding may allow for unimpeded circumferential expansion of the sinusoidal portion 1326.

Upon expansion of the expandable portion 1312 (by an expansion means such as a dilator or balloon—not shown) as seen in FIG. 71C, sinusoidal portion 1326 elongates in the circumferential direction, unimpeded by the embedding 1330. The overlap region 1332 of the outer layer 1322 allows it to expand. The overlap 1334 of the inner layer 1318 unfolds, resulting in an enlarged diameter for the expandable portion 1312, shown in FIG. 71C and FIG. 72.

All of the aspects described in the present disclosure (including references incorporated by reference, accompanying claims, abstract and drawings), may be combined in any order, in part or in full, or in any combination or modification, except when such are incompatible or inconsistent. Furthermore, each aspect may be replaced by alternative features serving the same, equivalent or similar purpose, unless expressly stated otherwise or inconsistent with the teachings herein. Thus, unless expressly stated otherwise, each aspect disclosed herein may be only an example of equivalent or similar features. It is intended that the invention be defined by the attached claims and their legal equivalents.

What is claimed is:

1. An introducer sheath system, comprising:
an expandable tubular sheath rolled along a longitudinal axis to define a lumen;
a hub assembly connected to a proximal end of the sheath; and
a dilator extending through the hub assembly and the sheath.

2. The introducer sheath of claim 1, wherein the tubular sheath includes an inner liner and an outer jacket.

3. The introducer sheath system of claim 2, wherein the outer jacket includes a longitudinal slit having edges, and a longitudinal hinge diametrically opposite the slit.

4. The introducer sheath system of claim 3, wherein the inner liner has a continuous circumference that is greater than a circumference of the outer jacket.

5. The introducer sheath of claim system 4, wherein the jacket is disposed coaxially over a portion of the inner liner to define a gap between the edges of the slit and a window of uncovered liner when the sheath is in an expanded state.

6. The introducer sheath system of claim 5, wherein the uncovered liner is folded inside toward the lumen and the edges of the slit come closer together to narrow or close the gap when the sheath is in a collapsed state.

7. The introducer sheath system of claim 6, wherein a temporary bond is made between the edges of the jacket at a distal end of the sheath.

8. The introducer sheath system of claim 7, wherein the folded liner is creased and crazed to retain a memory of its collapsed state.

9. The introducer sheath system of claim 8, wherein the tubular sheath includes a reinforcement structure disposed between the liner and the jacket, and wherein the reinforcement structure has a discontinuous circumference at the gap in the jacket.

10. The introducer sheath system of claim 9, wherein the reinforcement structure comprises a serpentine-shaped wire.

11. A guide extension catheter, comprising:
a push member having a proximal end connected to a hub and a distal end connected to a tubular section;
the tubular section having a proximal non-expandable portion and a distal expandable portion;
the non-expandable portion having a non-expandable lumen with a proximal opening and the expandable portion having an expandable lumen with a distal opening;
wherein the distal expandable portion is rolled along a longitudinal axis such that the expandable lumen is smaller than the non-expandable lumen when in a collapsed or non-expanded state.

12. The guide extension catheter of claim 11, wherein the tubular section includes an inner liner and an outer jacket.

13. The guide extension catheter of claim 12, wherein the outer jacket includes a longitudinal slit having edges, and a longitudinal hinge diametrically opposite the slit.

14. The guide extension catheter of claim 13, wherein the inner liner has a continuous circumference that is greater than a circumference of the outer jacket.

15. The guide extension catheter of claim 14, wherein the jacket is disposed coaxially over a portion of the liner to define a gap between the edges of the slit and a window of uncovered liner when in an expanded state.

16. The guide extension catheter of claim 15, wherein the uncovered liner is folded inside toward the lumen and the edges of the slit come closer together to narrow or close the gap when the distal expandable portion is in a collapsed state.

17. The guide extension catheter of claim 16, wherein a temporary bond is made between the edges of the jacket at a distal end of the expandable portion.

18. The guide extension catheter of claim 17, wherein the folded liner is creased and crazed to retain a memory of its collapsed state.

19. The guide extension catheter of claim 18, wherein the tubular section includes a reinforcement structure disposed between the liner and the jacket, wherein the reinforcement structure has a discontinuous circumference at the gap in the jacket, and wherein the reinforcement structure comprises a serpentine-shaped wire.

20. A guide extension catheter for use with a guide catheter and a guidewire, the guide extension catheter comprising:
a push member having a proximal end connected to a hub and a distal end connected to a tubular section;
the tubular section having a proximal non-expandable portion and a distal expandable portion;
the non-expandable having a non-expandable lumen with a proximal opening and the expandable portion having an expandable lumen with a distal opening;
wherein the distal expandable portion is rolled along a longitudinal axis such that the expandable lumen is smaller than the non-expandable lumen when in a collapsed or non-expanded state;
wherein the guide extension catheter is configured to have a length sufficient to extend through the guide catheter such that the hub may reside outside a proximal end of the guide catheter, the proximal opening may reside inside the guide catheter, and the expandable portion may reside outside a distal end of the guide catheter, with the guidewire extending out the proximal end of the guide catheter, through the tubular section, and out the distal opening of the expandable portion.
